(19) United States
(12) Patent Application Publication
(10) Pub. No.: US 2014/0357683 A1
(43) Pub. Date: Dec. 4, 2014

(54) METHODS OF TREATMENT AND COMPOSITIONS WITH XANTHINE OXIDASE INHIBITORS

(71) Applicants: TAKEDA PHARMACEUTICALS U.S.A., INC., Deerfield, IL (US); Teijin Pharma Limited, Tokyo (JP)

(72) Inventors: Lhanoo Gunawardhana, Pleasant Prairie, WI (US); Vijay Gupte, Vernon Hills, IL (US); Himanshu Naik, Evanston, IL (US); Michael Mayer, Antioch, IL (US); Kanji Komatsu, Tokyo (JP)

(21) Appl. No.: 14/292,010

(22) Filed: May 30, 2014

Related U.S. Application Data
(60) Provisional application No. 61/829,759, filed on May 31, 2013.
(60) Provisional application No. 61/839,609, filed on Jun. 26, 2013.

(51) Int. Cl. A61K 31/426; A61K 31/165

(52) U.S. Cl. CPC A61K 31/426; A61K 31/165
USPC 514/365

(57) ABSTRACT

Methods and pharmaceutical compositions for reducing number of gout flares experienced by a patient are disclosed. The methods can comprise administering to a patient with hyperuricemia an effective amount of a xanthine oxidase inhibitor in a modified release dosage form once daily or in an immediate release dosage form two or more times daily to prevent at least one gout flare or reduce the number of gout flares experienced by the patient.

### CROSS-REFERENCE TO RELATED APPLICATIONS

This application claims priority to U.S. Provisional Application Ser. No. 61/829,759, filed May 31, 2013, and to U.S. Provisional Application Ser. No. 61/839,609, filed Jun. 26, 2013, each of which is incorporated herein by reference in its entirety.

### BACKGROUND

Gout affects 3 to 5 million individuals in the United States and is increasing in incidence and prevalence. Gout is a serious health condition characterized by flares of acute arthritis, chronic gouty arthropathy, tophi, and uric acid urolithiasis, and is associated with a broad range of comorbidities, including cardiovascular (CV) disease, chronic kidney disease, and metabolic syndrome.

The underlying metabolic aberration in gout is hyperuricemia, in which the urate concentration in serum exceeds the limit of urate solubility (a serum urate (sUA) level of at least about 6.8 mg/dL for men). Hyperuricemia develops into gout when urate crystals are formed from supersaturated body fluids and deposited in joints, tophi, and parenchymal organs.

In humans and higher primates, uric acid is the final oxidation (breakdown) product of purine metabolism and is excreted in urine. Metabolic degradation of purines produces xanthine and hypoxanthine. The enzyme xanthine oxidase (XO) catalyzes the oxidation of hypoxanthine to xanthine and can further catalyze the oxidation of xanthine to uric acid.

Urate-lowering therapy (ULT) is used to treat hyperuricemia in subjects. Urate lowering therapy is recommended for subjects suffering from gout and one or more of the following conditions: acute gouty arthritis, chronic gouty joint disease, tophaceous gout, uric acid nephropathy, and/or nephrolithiasis (kidney stones).

In general, the goal of urate lowering therapy is to reduce sUA to below the concentration at which monosodium urate saturates extracellular fluid, 6.8 mg/dL. Using ULT to reduce and maintain sUA levels at less than 6.0 mg/dL or 5.0 mg/dL ultimately improves the clinical symptoms of gout by reducing the frequency of gout flares, decreasing size and number of tophi, and improving quality of life. Drugs that have been used in ULT include allopurinol, uricosuric drugs, and febuxostat.

Uricosuric drugs are substances that increase the excretion of uric acid in the urine, thus reducing the concentration of uric acid in blood plasma. Uricosuric drugs include as probenecid, benzbromarone and sulfinpyrazone. Use of these drugs is contraindicated in persons already with a high urine concentration of uric acid (hyperuricosuria).

Allopurinol and its metabolites are purine analogs. Therefore, in addition to inhibiting XO, allopurinol and its metabolites also inhibit other enzymes involved in purine and pyrimidine metabolism, increasing the potential for side effects.

In contrast, febuxostat (2-[3-cyano-4-(2-methylpropoxy)phenyl]-4-methylthiazole-5-carboxylic acid) is a potent nonpurine selective inhibitor of xanthine oxidase that exhibits anti-hyperuricemic activity by reducing formation of uric acid by XO. Febuxostat has been shown to potently inhibit both the oxidized and the reduced forms of XO. Febuxostat 40 and 80 mg once daily (QD) is approved in the United States for the chronic management of hyperuricemia in patients with gout.

Febuxostat is rapidly and well absorbed from the gastrointestinal tract after oral administration in animals. Febuxostat is almost entirely eliminated by liver metabolism, with <4% of orally administered febuxostat eliminated in the urine as unchanged drug. It is mainly metabolized by oxidation and/or glucuronidation, with glucuronidation as the major metabolic pathway in all species tested.

Extensive pharmacokinetic and pharmacodynamic data have established that maintaining a concentration of febuxostat in plasma over a prolonged period of time provides similar efficacy to treatment with high doses of the drug. Generally, these studies have shown that maintaining a febuxostat plasma concentration at or above 100 ng/ml results in about 80% or greater inhibition of xanthine oxidase. Therefore, a formulation of febuxostat that maintains the drug concentration at or above 100 ng/ml for an extended period of time is expected to result in higher efficacy of the drug, and would be a desirable treatment option for the control of hyperuricemia, gout, and many other disease states. However, currently, the only commercially available formulations of febuxostat are immediate release formulations. Although under development, no extended or delayed release formulations of febuxostat are commercially available at present.

Serum urate lowering therapy is associated with an increased frequency of acute gout flares. A gout flare is a sudden attack of intense pain and swelling in the affected joint(s). Decreases in serum urate are thought to cause transient localized precipitation of monosodium urate crystals in cartilage and soft tissues, leading to acute gout flares. In two studies of ULT with data available on patients not taking a form of prophylaxis against gout flares, the frequency of acute flares accompanying initiation of ULT was 38% and 75%. The increased incidence of intensely painful gout flares with new ULT treatment can affect patient compliance with the new ULT treatment regimen. (In some cases, the patient will cease ULT therapy because of gout flares. Harrold L R, Andrade S E, Briesacher B A, Raebel M A, Fouayzi H, Yood R A, et al. Adherence with urate-lowering therapies for the treatment of gout. Arthritis Res Ther. 2009; 11:R46.)

In some markets, and for specific populations, a dose-escalating (i.e. dose titration) regimen is recommended to prevent acute gout flares in the serum urate lowering therapy. For example, in Japan, Feburic® Tablet is marketed as a once daily febuxostat immediate release formulation where the usual adult dose is once daily starting from 10 mg and after that the dose is increased gradually, with the usual maintenance dose from 40 mg once daily.

In a clinical trial, patients initiating allopurinol urate lowering treatment who received colchicine as prophylaxis against gout flares for the first six months of allopurinol treatment experienced fewer total flares and less severe flares than patients who did not receive any prophylactic treatment (Borstad, G C et al. J Rheumatol 2004; 31; 2429-2432). Anti-inflammatory agents and/or colchicine are frequently given as prophylaxis for gout flares during the first months of ULT treatment. Despite the effectiveness of anti-inflammatory agents and/or colchicine at reducing the number or degree and severity of gout flares, a number of patients taking these adjunctive therapies will experience side effects of those drugs. In some cases, the patient will cease adjunctive therapies because of side effects or has to take lower doses of adjunctive therapies due to co-morbid conditions or due to potential drug drug interactions. Further, some patients are unable to take these adjunctive medications as they may be contraindicated due to certain medical conditions.

There remains a need in the art for improved methods of reducing the incidence of gout flares associated with initiating ULT treatment.

### SUMMARY

Methods of preventing at least one gout flare or reducing the number or degree of gout flares experienced by a patient are disclosed herein. Also disclosed herein are methods of treatment with a xanthine oxidase inhibitor in a dosing regimen which is a non-dose-escalating regimen, where the level of gout flare rate or degree is similar to level of gout flare rate or degree for a dose-escalating regimen.

In an embodiment, the method comprises administering to a patient with hyperuricemia an effective amount of a xanthine oxidase inhibitor in a modified release dosage form once daily or in an immediate release dosage form two or more times daily to prevent at least one gout flare or reduce the number or degree of gout flares experienced by the patient, wherein the xanthine oxidase inhibitor is febuxostat, topiroxostat (4-[45-(pyridin-4-yl)-1H-1,2,4-triazol-3-yl]pyridine-2-carbonitrile), allopurinol, a compound described or claimed in U.S. Pat. No. 7,598,254 (WO2005/121153) or US2012015972 (WO2010/113942), or a triarylcarboxylic acid compound described or claimed in U.S. Pat. No. 7,816,558 (WO2007/043457) or represented by the following formula (I) or a salt thereof:

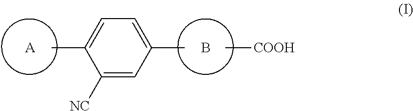

wherein: A: aryl or heteroaryl, wherein aryl and heteroaryl may be substituted with the same or different, 1 to 3 substituents selected from the following group G;

group G: halogen, —CN, —NO₂, lower alkyl, halogeno-lower alkyl, —O—R¹, —O-halogeno-lower alkyl, —O—CO—R′, —O-benzyl, —O-phenyl, —NR²R³, —CO—NR²R³, —CO-phenyl, —S—R′, —SO₂-lower alkyl, —SO₂-phenyl, —NH—SO₂-naphthalene-NR²R³, phenyl, cycloalkyl, and -lower alkylene-O—R¹;

R¹: H or lower alkyl;

R² and R³: same or different, each representing H or lower alkyl,

wherein R² and R³, taken together with the nitrogen atom to which they bond, may form a monocyclic nitrogen-containing saturated heterocycle; and

B: monocyclic heteroaryl, wherein the monocyclic heteroaryl may be substituted with a group selected from lower alkyl, —OH, and halogen.

In an embodiment, the method comprises preventing at least one gout flare or reducing the number or degree of gout flares experienced by a patient by administering to a patient with hyperuricemia an effective amount of a xanthine oxidase inhibitor in a modified release dosage form once daily or in an immediate release dosage form two or more times daily.

Methods of preserving renal function of a patient are disclosed herein.

In an embodiment, the method comprises administering to a patient with hyperuricemia an effective amount of a xanthine oxidase inhibitor in a modified release dosage form once daily or in an immediate release dosage form two or more times daily to preserve renal function of the patient.

In an embodiment, the method comprises preserving renal function of a patient by administering to a patient with hyperuricemia an effective amount of a xanthine oxidase inhibitor in a modified release dosage form once daily or in an immediate release dosage form two or more times daily.

Also disclosed herein are methods of treating a patient with a xanthine oxidase inhibitor.

In an embodiment, the method comprises administering to a patient in need thereof an effective amount of a xanthine oxidase inhibitor in a modified release dosage form once daily or in an immediate release dosage form two or more times daily, wherein during xanthine oxidase inhibitor administration the number or degree of gout flares characterizing once daily administration of the modified release dosage form or twice daily administration of the immediate release dosage form of the xanthine oxidase inhibitor is reduced from the number or degree of gout flares characterizing once daily administration of an immediate release dosage form of the xanthine oxidase inhibitor.

In an embodiment, the method comprises administering to a patient in need thereof an effective amount of a xanthine oxidase inhibitor in a modified release dosage form once daily or in an immediate release dosage form two or more times daily, wherein during xanthine oxidase inhibitor administration the number or degree of gout flares characterizing once daily administration of the modified release dosage form or twice daily administration of the immediate release dosage form of the xanthine oxidase inhibitor is less than or equal to the number or degree of gout flares characterizing administration of placebo.

In an embodiment, the method comprises administering to a patient in need thereof an effective amount of a xanthine oxidase inhibitor in a modified release dosage form once daily or in an immediate release dosage form two or more times daily, wherein during xanthine oxidase inhibitor administration once daily administration of the modified release dosage form or twice daily administration of the immediate release dosage form of the xanthine oxidase inhibitor preserved renal function better than once daily administration of an immediate release dosage form of the xanthine oxidase inhibitor.

In an embodiment, the method comprises administering to a patient in need thereof an effective amount of a xanthine oxidase inhibitor in a modified release dosage form once daily or in an immediate release dosage form two or more times daily, wherein during xanthine oxidase inhibitor administration once daily administration of the modified release dosage form or twice daily administration of the immediate release dosage form of the xanthine oxidase inhibitor preserved renal function better than did administration of placebo.

In an embodiment, the method comprises administering an effective amount of a xanthine oxidase inhibitor in a modified release dosage form once daily for the chronic management of hyperuricemia in patients with gout in order to achieve a reduction in the frequency of gout flares compared with immediate release dosage forms of xanthine oxidase inhibitor.

Pharmaceutical compositions containing a xanthine oxidase inhibitor for preventing at least one gout flare or reducing the number or degree of gout flares experienced by a patient are also disclosed. Also disclosed herein are pharmaceutical compositions containing a xanthine oxidase inhibitor which is a non-dose-escalating regimen, where the level of gout flare rate or degree is similar to level of gout flare rate in a dose-escalating regimen.

In an embodiment, the pharmaceutical composition is a modified release dosage form for once daily administration.

In an embodiment, the pharmaceutical composition is an immediate release dosage form for at least twice daily administration.

These and other embodiments, advantages and features of the present invention become clear when detailed description and examples are provided in subsequent sections.

### BRIEF DESCRIPTION OF THE DRAWINGS

FIG. 1 is a histogram showing the percentage of subjects in the three treatment groups (placebo, 40/80 mg once daily (QD) immediate release febuxostat, 30 mg twice daily (BID) immediate release febuxostat) with a serum uric acid (sUA) level of <6 mg/dL at 6 and 12 months, respectively. The symbol *** indicates statistical significance at <0.001 level, for a comparison with the placebo group.

FIG. 2 is a histogram showing the percentage of subjects with gout flares in the three treatment groups (placebo, 40/80 mg once daily (QD) febuxostat, 30 mg twice daily (BID) febuxostat) for the first 6 months and the second six months of the trial, respectively.

FIG. 3 shows a graph of mean plasma febuxostat (ULORIC®) concentration (μg/mL) at steady state (Day 14) as a function of time following administration to healthy subjects of 120 mg immediate release (IR) QD, 30 mg IR BID, or 80 mg extended release (XR) QD and simulated steady state results for administration of 40 mg XR QD to healthy subjects.

FIG. 4 is a histogram showing the Mean Change in eGFR from Baseline at Month 6 and Month 12.

FIG. 5 is a histogram showing the Mean Change from Baseline in eGFR at Month 6 (M6) and Month 12 (M12) by Baseline Renal Function.

FIG. 6 is a histogram showing the Mean Change from Baseline in eGFR at Month 6 (M6) and Month 12 (M12) by Baseline ARB and ACEi Use.

FIG. 7 is a schematic illustration of febuxostat IR and DR6.8 beads.

FIG. 8 shows graphs of dissolution profiles of febuxostat XR (panel XR (40 mg) for 40 mg febuxostat and panel XR (80 mg) for 80 mg febuxostat), Formulations B (panel A), C (panel B), D (panel C), and E (panel D), and CR-long beads (panel E) determined by the dissolution test method for these formulations described in Example 5.

FIG. 9 shows graphs of dissolution profiles of Formulations 1, 2, 3, and 4, determined by the dissolution test method for these formulations described in Example 5.

### DETAILED DESCRIPTION

Disclosed herein are methods, pharmacokinetic profiles, and compositions for reducing incidence or degree of gout flares. The methods, pharmacokinetic profiles, and compositions permit reduction in the number or degree of gout flares associated with initiation of urate lowering therapy (ULT) in patients in need thereof. Methods, pharmacokinetic profiles, and compositions disclosed also permit reduction in the number or degree of gout flares associated with initiation of ULT without a loss of ULT efficacy. Methods, non-dose-escalating dosing regimens, pharmacokinetic profiles, and compositions disclosed herein further permit ULT in patients without the need for a dose-escalating regimen since the methods result in a same or similar incidence or degree of gout flares compared with dose-escalating regimens.

Serum urate lowering is associated with an increased frequency of acute gout flares, especially in the early stages of initiating ULT. In two studies, with data available on patients initiating ULT and not taking a form of prophylaxis against gout flares, the frequency of gout flares during initiation of ULT was 38% and 75%. (Borstad, G C et al. J Rheumatol 2004; 31; 2429-2432) Decreases in serum urate are thought to cause mobilization of monosodium urate crystals in joints, leading to these treatment-initiated gout flares. Therefore, a ULT treatment with better efficacy early in the treatment is expected to have a higher incidence of ULT-initiated flares.

The European League Against Rheumatism (EULAR) gout task force has recommended that allopurinol ULT be started at low doses and increased over several weeks, with the aim of lowering urate concentrations slowly to minimize risk of acute flare episodes. (Zhang, W. et al., Ann Rheum Dis 2006, 65: 1312-1324.), EULAR also recommends that, at the same time as initiating ULT, either colchicine or a low dose NSAID be prescribed for up to at least the first 6-months of ULT for prophylaxis to prevent/reduce flares. (Zhang, W. et al., Ann Rheum Dis 2006, 65: 1312-1324) It has been reported that gout flare rates increased sharply in the period immediately after prophylaxis withdrawal during ULT (Becker M A, et al., J Rheumatol. 2009 June; 36(6):1273-82).

Febuxostat exhibits anti-hyperuricemic activity. Unlike allopurinol, febuxostat is a nonpurine selective inhibitor of xanthine oxidase. Pharmacokinetic and pharmacodynamic studies with febuxostat have established that maintaining a concentration of febuxostat in plasma over a prolonged period of time provides similar efficacy to treatment with high doses of the febuxostat. Generally, these studies have shown that maintaining a febuxostat plasma concentration of 100 ng/ml is required to provide 95% or greater inhibition of xanthine oxidase. Therefore, a febuxostat dosage form or a febuxostat dosing regimen that maintains the drug concentration at or above 100 ng/ml for an extended period of time is expected to result in higher efficacy of the drug, and would be a desirable treatment option for the control of hyperuricemia, gout, and many other disease states. However, due to its enhanced efficacy, such a febuxostat dosing regimen or dosage form was expected to be associated with increased acute gout flares during the early treatment period.

It has been unexpectedly discovered that certain febuxostat dosing regimens result in a significant reduction of the number or degree/percentage of subjects with gout flares, while achieving greater serum urate reduction, compared to once daily administration of 40 mg or 80 mg immediate release febuxostat formulations. The febuxostat dosing regimens also result in a significant reduction of the number or degree/percentage of subjects with gout flares (e.g. based on a mean value, median value, etc.), while achieving greater serum urate reduction, compared to once daily administration of the immediate release dosage form. The febuxostat dosing regimens show equivalent or similar serum urate reduction efficacy as the once daily administration of the immediate release dosage form. Additionally, the number or degree/percentage of subjects with gout flares in the group receiving the febuxostat dosing regimens did not increase significantly compared to the number or degree/percentage of subjects with gout flares in a placebo group. In particular, the number or degree/percentage of subjects with gout flares in the group receiving the febuxostat dosing regimens did not increase significantly compared to the number or degree/percentage of subjects with gout flares in the placebo group after cessation of concomitant gout flare prophylactic treatment with the febuxostat dosing regimen. In contrast, the number or degree/percentage of subjects with gout flares in the group receiving once daily administration of a febuxostat immediate release formulation, e.g. a 40 or 80 mg febuxostat formulation, increased markedly compared to the number or degree/percentage of subjects with gout flares in the placebo group after cessation of concomitant gout flare prophylactic treatment with the febuxostat dosing regimen. Further the febuxostat dosing regimens show equivalent or similar efficacy in reduction of the number or degree/percentage of subjects with gout flare or hyperurecemia as the dose-escalating regimen. Further it is considered that the febuxostat dosing regimens result in a significant extension of the time to first new gouty attack of subjects with gout or hyperuricemia compared to the time in the group receiving once daily administration of a febuxostat immediate release formulation or in the group receiving the dose-escalating regimens or in the placebo group.

While not wanting to be bound by theory, delivery of an amount of ULT in a modified dosage form is believed to reduce the risk of a patient experiencing a gout flare due to a gentler reduction in serum uric acid levels. Following the discoveries described herein, it is believed that daily fluctuations in ULT may lead to an increase in gout flares as serum urate levels crash rapidly in the blood stream. In many gout patients with hyperurcemia, tophi—deposits of uric acid crystals—form in joints such as the hands or feet, ear, elbow, or Achilles tendon. Gout flares are believed to be caused in part by the mobilization of uric acid crystals in affected joints. The serum urate level crash resulting from effective urate lowering therapy creates a higher concentration gradient between the location of urate crystals and the bloodstream, thereby causing a more rapid mobilization of crystals and a resulting gout flare.

For example, an 80 mg dose of febuxostat delivered in a modified release formulation will reduce gout flares compared to an 80 mg dose of febuxostat delivered in an immediate release formulation. Several metrics can be used to describe the pharmacokinetic characteristics of formulations that will likely achieve the same result of maintaining active levels of drug over a longer period of time while reducing total drug exposure while achieving equivalent reduction of sUA, for example, the parameters Cₘₐₓ/dose, Mean Residence Time, Cₘₐₓ/Cₘᵢₙ, AUC₀₋₄, AUC₄₋₂₄, AUC₂₄/dose, Tₘₐₓ. The foregoing pharmokinetic metrics are generally discussed in terms of the mean values.

It has also been unexpectedly discovered that the xanthine oxidoreductase inhibitor formulations characterized by certain pharmacokinetic parameters result in a significant reduction of the number or degree/percentage of subjects with gout flares. More concretely, xanthine oxidoreductase inhibitor formulations which, after administration to a subject in need thereof, produce fluctuations in the subject's plasma concentration profile of the xanthine oxidoreductase inhibitor within a certain value for a period after administration up to 24 hours, result in a significant reduction of the number or degree/percentage of subjects with gout flares. More concretely, xanthine oxidoreductase inhibitor formulations which, after administration to a subject in need thereof, produce in the subject a ratio of maximum plasma concentration (Cₘₐₓ) to minimum plasma concentration (Cₘᵢₙ) at steady state of the xanthine oxidoreductase inhibitor less than or equal to 60 for a period of from administration to 24 hours, result in a significant reduction of the number or degree/percentage of subjects with gout flares.

It has been unexpectedly discovered that modified release formulations of 80 mg febuxostat showed lower incidence of gout flares compared to an 80 mg febuxostat immediate release formulation. The modified release formulations of 80 mg febuxostat were characterized in pharmacokinetic studies to determine the pK parameters associated with the lower incidence of gout flares during administration, as discussed further below.

Also disclosed herein are methods of preserving renal function of a patient. These methods provide improved preservation of renal function during urate lowering therapy (ULT) in patients in need thereof.

It has been unexpectedly discovered that certain febuxostat dosing regimens result in improved preservation of renal function compared to once daily administration of 40 mg or 80 mg immediate release febuxostat formulations. Additionally, subjects receiving the febuxostat dosing regimens improved preservation of renal function compared to subjects receiving placebo.

The febuxostat dosing regimen can be administration once daily of a febuxostat extended release dosage form, for example having 1-120 mg febuxostat, specifically 1-80 mg febuxostat, specifically 1-40 mg febuxostat, or administration at least twice daily of an immediate release febuxostat dosage form, for example having 1-120 mg febuxostat, specifically 1-80 mg febuxostat, specifically 1-40 mg febuxostat. The febuxostat can be present in the dosage form at about 1 mg to about 500 mg, about 1 mg to about 240 mg, about 1 mg to about 120 mg, about 1 mg to about 80 mg, or about 1 mg to about 40 mg. For example, the modified release dosage form or the immediate release dosage form used in the methods can contain about 5 mg, about 10 mg, about 20 mg, about 30 mg, about 40 mg, about 50 mg, about 60 mg, about 70 mg, about 80 mg, about 90 mg, about 100 mg, about 110 mg, or about 120 mg febuxostat. In certain embodiments, an oral modified release dosage form has 40 mg or 80 mg of febuxostat. In certain embodiments, an oral immediate release dosage form has 30 mg of febuxostat. In certain embodiments, an oral immediate release dosage form has 120 mg of febuxostat In some embodiments, the amount of febuxostat in the dosage form is about 1 mg to about 500 mg, about 1 mg to about 240 mg, about 5 mg to about 120 mg, about 5 mg to about 80 mg, about 10 mg to about 50 mg. The modified release dosage form or the immediate release dosage form is an oral dosage form.

Xanthine oxidase inhibitors other than febuxostat are expected to be characterized by analogous effects to those disclosed for febuxostat. Other xanthine oxidase inhibitors include topiroxostat, allopurinol, a compound described or claimed in U.S. Pat. No. 7,598,254 (WO2005/121153) or US2012015972 (WO2010/113942), or a triarylcarboxylic acid compound described or claimed in U.S. Pat. No. 7,816,558 (WO2007/043457) or represented by the following formula (I) or a salt thereof:

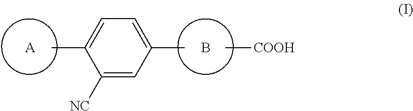

wherein: A: aryl or heteroaryl, wherein aryl and heteroaryl may be substituted with the same or different, 1 to 3 substituents selected from the following group G;

group G: halogen, —CN, —NO2, lower alkyl, halogeno-lower alkyl, —O—R1, —O-halogeno-lower alkyl, —O—CO—R1, —O-benzyl, —O-phenyl, —NR2R3, —NH—CO—R1, —CO2-R1, —CO—R1, —CO—NR2R3, —CO-phenyl, —S—R1, —SO2-lower alkyl, —SO2-phenyl, —NH—SO2-naphthalene-NR2R3, phenyl, cycloalkyl, and -lower alkylene-O—R1;

R1: H or lower alkyl;

R2 and R3: same or different, each representing H or lower alkyl,

wherein R2 and R3, taken together with the nitrogen atom to which they bond, may form a monocyclic nitrogen-containing saturated heterocycle; and

B: monocyclic heteroaryl, wherein the monocyclic heteroaryl may be substituted with a group selected from lower alkyl, —OH, and halogen. U.S. Pat. No. 7,598,254 (WO2005/121153), US2012015972 (WO2010/113942), and U.S. Pat. No. 7,816,558 (WO2007/043457) are incorporated by reference herein in their entirety. In the above formula (I), the definitions of the substituents are the same as those specified in U.S. Pat. No. 7,816,558 and international patent application WO2007/043457.

A compound described or claimed in U.S. Pat. No. 7,598,254 (WO2005/121153) is represented by the following formula or a salt thereof:

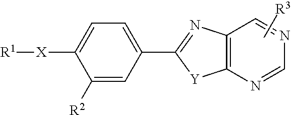

wherein R1 represents an aryl group having 6-10 carbon atoms or a hetero-aryl group which may have a substituent selected from the group and atom consisting of an alkyl group having 1-8 carbon atoms, a halogen-substituted alkyl group having 1-8 carbon atoms, an alkoxy group having 1-8 carbon atoms, an alkoxy group having 1-8 carbon atoms which is substituted with an alkoxy group having 1-8 carbon atoms, an alkoxycarbonyl group having 2-8 carbon atoms, formyl, carboxyl, halogen, hydroxyl, nitro, cyano, amino, an aryl group having 6-10 carbon atoms, and an aryloxy group having 6-10 carbon atoms;
R2 represents cyano, nitro, formyl, carboxyl, carbamoyl, or an alkoxycarbonyl group having 2-8 carbon atoms;
R3 represents hydroxyl, amino, carboxyl, mercapto, OR4 or NHR5 in which each of R4 and R5 is an alkyl group having 1-8 carbon atoms which may have a substituent selected from the group and atom consisting of halogen, hydroxyl, nitro, cyano, amino, an aryl group having 6-10 carbon atoms, and an aryloxy group having 6-10 carbon atoms;
X represents oxygen, —N(R6)-, or —S(O)n- in which R6 is hydrogen, an alkyl group having 1-8 carbon atoms, or the group for R1, and n is an integer of 0 to 2; and
Y represents oxygen or sulfur.

A compound described or claimed in US2012015972 (WO2010/113942) is represented by the following formula or prodrug thereof, or a pharmaceutically acceptable salt thereof:

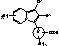

wherein ring U represents aryl or heteroaryl;
R1 represents a halogen atom, a hydroxy group, nitro, amino or C1-6 alkyl which may be substituted by a fluorine atom;
R2 represents any of the following (1) to (7):(1) a halogen atom; (2) a hydroxy group; (3) amino; (4) carbamoyl; (5) cyano; (6) carboxy; (7) C1-6 alkyl, C2-6 alkenyl, C2-6 alkynyl, C1-6 alkoxy, mono(di) C1-6 alkylamino, C2-7 acyl, C2-7 acylamino, mono(di)C1-6alkylcarbamoyl, C1-6 alkylsulfonyl, C1-6 alkylsulfonylamino, mono(di)C1-6alkylsulfamoyl, C1-6 alkylthio, C2-6 allcenyl C1-6 alkoxy, C3-8 cycloalkyl, 3 to 8-membered heterocycloalkyl, C5-8 cycloalkenyl, 5 to 8-membered heterocycloalkenyl, C3-8 cycloalkyloxy, C3-8 cycloalkylamino, C3-8 cycloalkyl C1-6 alkyl, C3-8 cycloalkyl C1-6 alkoxy, C3-8 cycloalkyl C1-6 alkylamino, aryl, heteroaryl, aryloxy, arylamino, arylcarbonyl, arylcarbonylamino, arylC1-6 alkoxy, heteroaryloxy, heteroarylamino, heteroarylcarbonyl or heteroarylcarbonylamino each of which may have any group selected from substituent group a;
m represents an integral number from 0 to 2, and when m is 2, these R1 are optionally different from each other;
n represents an integral number from 0 to 3, and when n is 2 or 3, these R2 are optionally different from each other; and when two R2 bound to the neighboring atoms in the indolizine ring exist and independently represent a group selected from the group consisting of C1-6 alkyl which may be substituted by a fluorine atom and C1-6 alkoxy which may be substituted by a fluorine atom, these two R2 optionally form a 5 to 8-membered ring together with the binding atoms in the indolizine ring;
R3 represents a hydrogen atom, a chlorine atom or a fluorine atom; and substituent group a consists of a fluorine atom, a chlorine atom, a hydroxy group, amino, carboxy, carbamoyl, cyano, C1-6 alkyl, C1-6 alkoxy and mono(di)C1-6 alkylamino.

“Concomitant” and “concomitantly” as used herein refer to the administration of at least two active agents to a patient either simultaneously or within a time period during which the effects of the first administered active agent are still operative in the patient.

The terms “prophylaxis”, “prophylactic treatment”, and “prophylactic” with respect to gout flares mean a measure to ward off or avoid occurrence of a gout flare in a subject or patient who has risk of a gout flare, or to lower the risk or frequency of experiencing a gout flare of a subject or patient. For example, a prophylactic treatment against gout flares can be administration of an anti-inflammatory such as colchicine or a non-steroidal anti-inflammatory (NSAID) including, for example, indomethacin, naproxen, oxaprozin, pranoprofen, diclofenac or loxoprofen in an effective amount for the patient. For example, 0.6 mg colchicine can be administered once daily or every other day as prophylaxis against gout flares during ULT, especially during the initial period of ULT when treatment-initiated gout flares may occur.

Herein, “initiation of urate-lowering therapy” refers to administration of a first dose of a urate-lowering pharmaceutical composition to a subject to whom no urate lowering therapy has been administered during the 14 days previous to administration of the first dose of the urate-lowering pharmaceutical composition, specifically during the 21 days previous to administration of the first dose of the urate-lowering pharmaceutical composition, more specifically during the 30 days previous to administration of the first dose of the urate-lowering pharmaceutical composition.

The “initial stage” or “initial period” of ULT refers to the first 12 months, the first 6 months, the first 5 months, the first 4 months, the first 3 months, the first two months, the first month, or the first two weeks of ULT after initiation of the ULT.

A “treatment-initiated gout flare” refers to a gout flare occurring during the initial period of ULT.

Herein, a “gout flare” generally means a patient-reported acute articular pain typical of a gout attack that is deemed by the patient and/or a medical care provider to require treatment and includes at least three or more of joint swelling, redness, tenderness, and pain and at least one or more of rapid onset of pain, decreased range of motion, joint warmth, and symptoms similar to a prior gout flare. Occurrence of gout flares can be documented for determination of incidence using an assessment worksheet for completion by a patient and/or by a medical care provider. Typically, a gout flare patient self-assessment worksheet requests information regarding site of the gout flare, signs/symptoms, relative strength of the signs/symptoms, and a pain rating for the flare. Additionally information regarding treatment may be collected. Optionally a medical care provider may provide an opinion regarding the likelihood that the patient self-assessed episode was a true gout flare.

Herein, a “degree of gout flares” means relative strength of the signs/symptoms and/or the pain rating for the gout flares.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism.

Disclosed herein are methods of preventing at least one gout flare or reducing the number or degree of gout flares experienced by a patient.

The terms “prevent”, “preventing”, and “preventive treatment” with respect to gout flares mean to ward off or avoid occurrence of at least one gout flare in a subject or patient who has risk of a gout flare, or to lower the risk or frequency of experiencing a gout flare of a subject or patient.

In an embodiment, the method comprises administering to a patient with hyperuricemia a xanthine oxidase inhibitor in a modified release dosage form once daily or in an immediate release dosage form two or more times daily to prevent at least one gout flare or reduce the number or degree of gout flares experienced by the patient.

In an embodiment, the method comprises preventing at least one gout flare or reducing the number or degree of gout flares experienced by a patient by administering to a patient with hyperuricemia a xanthine oxidase inhibitor in a modified release dosage form once daily or in an immediate release dosage form two or more times daily.

In an embodiment, the method comprises orally administering to a patient with hyperuricemia an effective amount of febuxostat in a modified release dosage form once daily to prevent at least one gout flare or reduce the number or degree of gout flares experienced by the patient, the modified release dosage form providing, after administration of a single dose, a mean residence time (MRTinf) of the febuxostat of at least 7 hours. In some embodiments, the MRTinf is at least 8 hours, at least 9 hours, at least 10 hours, at least 11 hours, or at least 12 hours. In an embodiment, the MRTinf has a value between about 7 hours and about 16 hours, about 8 hours and about 15 hours, about 9 hours and about 14 hours, about 10 hours and about 13 hours, or about 11 hours and about 13 hours. In an embodiment, the MRTinf about 12 hours.

In an embodiment, the method comprises orally administering to a patient with hyperuricemia an effective amount of febuxostat in a modified release dosage form once daily to prevent at least one gout flare or reduce the number or degree of gout flares experienced by the patient, the modified release dosage form providing, after administration of a single dose, a Cmax per dose strength of less than about 20 ng/mL/mg. In some embodiments, the Cmax per dose strength is less than about 19 ng/mL/mg, less than about 18 ng/mL/mg, less than about 17 ng/mL/mg, less than about 16 ng/mL/mg, less than about 15 ng/mL/mg, less than about 14 ng/mL/mg, or less than about 13 ng/mL/mg. In an embodiment, the Cmax per dose strength is between about 11 ng/mL/mg. to about 13 ng/mL/mg.

In an embodiment, the method comprises orally administering to a patient with hyperuricemia 80 mg febuxostat in a modified release dosage form once daily to prevent at least one gout flare or reduce the number or degree of gout flares experienced by the patient, the modified release dosage form providing, after administration of a single dose, a Cmax of less than about 1500 ng/mL. In an embodiment, Cmax is less than about 1400 ng/mL, less than about 1200 ng/mL, less than about 1100 ng/mL, or less than about 1000 ng/mL. In an embodiment, Cmax is in the range of about to about 900 ng/ml to about 1500 ng/ml. In an embodiment, the Cmax is in the range of about 950 ng/ml to about 1450 ng/ml, or about 980 ng/ml to about 1400 ng/ml.

In an embodiment, the method comprises orally administering to a patient with hyperuricemia 40 mg febuxostat in a modified release dosage form once daily to prevent at least one gout flare or reduce the number or degree of gout flares experienced by the patient, the modified release dosage form providing, after administration of a single dose, a Cmax of less than about 750 ng/mL. In an embodiment, Cmax is less than about 700 ng/mL, less than about 600 ng/mL, less than about 550 ng/mL, or less than about 500 ng/mL. In an embodiment, Cmax is in the range of about to about 450 ng/ml to about 750 ng/ml. In an embodiment, the Cmax is in the range of about 475 ng/ml to about 725 ng/ml, or about 490 ng/ml to about 700 ng/ml.

In an embodiment, the method comprises orally administering to a patient with hyperuricemia an effective amount of febuxostat in a modified release dosage form once daily to prevent at least one gout flare or reduce the number or degree of gout flares experienced by the patient, the modified release dosage form providing, after administration of a single dose, a Tmax in the range of about 2 hours to about 8 hours. In an embodiment, Tmax is in the range of about 3 hours to about 7 hours, about 4 hours to about 7 hours, about 5 hours to about 7 hours. In an embodiment, Tmax is about 6 hours.

In an embodiment, the method comprises orally administering to a patient with hyperuricemia an effective amount of febuxostat in a modified release dosage form once daily to prevent at least one gout flare or reduce the number or degree of gout flares experienced by the patient, the modified release dosage form providing, after administration of a single dose, an area under the curve from time 0 to 4 hours (AUC₀₋₄) of less than about 1800 hr-ng/mL. In an embodiment, AUC₀₋₄ is less than about 1800 hr-ng/mL, about 1600 hr-ng/mL, about 1400 hr-ng/mL, about 1200 hr-ng/mL, or about 1000 hr-ng/mL. In an embodiment, AUC₀₋₄ is in a range of about 800 hr-ng/mL to about 2000 hr-ng/mL. In an embodiment, AUC₀₋₄ is in a range of about 850 hr-ng/mL to about 1800 hr-ng/mL, about 900 hr-ng/mL to about 1600 hr-ng/ml, about 900 hr-ng/mL to about 1400 hr-ng/ml, about 900 hr-ng/mL to about 1200 hr-ng/ml.

In an embodiment, the method comprises orally administering to a patient with hyperuricemia an effective amount of febuxostat in a modified release dosage form once daily to prevent at least one gout flare or reduce the number or degree of gout flares experienced by the patient, the modified release dosage form providing, after administration of a single dose, an area under the curve from time 4 hours to time 24 hours (AUC₄₋₂₄) is more than about 4000 hr-ng/mL. In an embodiment, AUC₄₋₂₄ is more than about 4100 hr-ng/mL, about 4200 hr-ng/mL, about 4300 hr-ng/mL, about 4400 hr-ng/mL, about 4500 hr-ng/mL, about 4500 hr-ng/mL, or about 4700 hr-ng/mL. In an embodiment, AUC₄₋₂₄ is in a range of about 4000 hr-ng/mL to about 5000 hr-ng/mL, about 4200 hr-ng/mL to about 4900 hr-ng/mL, about 4400 hr-ng/mL to about 4900 hr-ng/mL, or about 4600 hr-ng/mL to about 4900 hr-ng/mL.

In an embodiment, the method comprises administering an effective amount of a xanthine oxidase inhibitor in a modified release dosage form once daily for the chronic management of hyperuricemia in patients with gout in order to achieve a reduction in the frequency of gout flares compared with immediate release dosage forms of xanthine oxidase inhibitor.

In any of the above embodiments, the effective amount is about 40 mg or about 80 mg. In any of the above embodiments, the effective amount is about 80 mg.

In any embodiment of these methods, preventing at least one gout flare or reducing the number or degree of gout flares experienced by the patient can occur during an initial period of administration of the xanthine oxidase inhibitor.

Methods of reducing incidence of gout flares associated with initiation of urate-lowering therapy with febuxostat are disclosed. In an embodiment, the method comprises administering a febuxostat modified release dosage form once daily or a febuxostat immediate release dosage form at least twice daily to a patient in need of initiating urate-lowering therapy. The amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, or about 1 mg to about 40 mg. For example, the modified release dosage form or the immediate release dosage form used in the methods can contain about 5 mg, about 10 mg, about 20 mg, about 30 mg, about 40 mg, about 80 mg, or about 120 mg febuxostat. In certain embodiments, an oral modified release dosage form has 40 mg or 80 mg of febuxostat. In certain embodiments, an oral immediate release dosage form has 30 mg of febuxostat. In certain embodiments, an oral immediate release dosage form has 120 mg of febuxostat. In some embodiments, the amount of febuxostat in the dosage form is about 1 mg to about 500 mg, about 1 mg to about 240 mg, about 5 mg to about 120 mg, about 5 mg to about 80 mg, about 10 mg to about 50 mg. The modified release dosage form or the immediate release dosage form is an oral dosage form.

Methods of treating a patient with a xanthine oxidase inhibitor are also disclosed.

In an embodiment, the method comprises administering to a patient in need thereof a xanthine oxidase inhibitor in a modified release dosage form once daily or in an immediate release dosage form two or more times daily. The amount of the xanthine oxidase inhibitor in the dosage form can be an effective amount.

In an embodiment, the method comprises administering a febuxostat modified release dosage form once daily or a febuxostat immediate release dosage form at least twice daily to a patient in need thereof, wherein the amount of febuxostat in the dosage form is about 5 mg to about 120 mg febuxostat. During the initial period of ULT, the number or degree of subjects having gout flares characterizing once daily administration of a febuxostat modified release dosage form or at least twice daily administration of a febuxostat immediate release dosage form is reduced from the number or degree of subjects having gout flares characterizing once daily administration of a febuxostat immediate release dosage form containing 40 mg or 80 mg febuxostat. During the initial period of ULT, the number or degree of subjects having gout flares and receiving once daily administration of a febuxostat modified release dosage form or at least twice daily administration of a febuxostat immediate release dosage form is reduced from the number or degree of subjects having gout flares and receiving once daily administration of an immediate release dosage form of febuxostat, wherein the once daily administration of the modified release dosage form or twice daily administration of the immediate release dosage form shows equivalent or similar serum urate reduction efficacy as the once daily administration of an immediate release dosage form. During the initial period of ULT, the degree of severity of gout flares of subjects receiving once daily administration of a febuxostat modified release dosage form or at least twice daily administration of a febuxostat immediate release dosage form is reduced from the the degree of severity of gout flares of subjects receiving once daily administration of an immediate release dosage form of febuxostat, wherein the once daily administration of the modified release dosage form or twice daily administration of the immediate release dosage form shows equivalent or similar serum urate reduction efficacy as the once daily administration of an immediate release dosage form. Further, during the initial period of ULT, once daily administration of a febuxostat modified release dosage form or at least twice daily administration of a febuxostat immediate release dosage form is characterized by an incidence of gout flares that is less than or equal to incidence of gout flares characterizing administration of placebo.

The methods are characterized by use of xanthine oxidase inhibitor formulations characterized by certain pharmacokinetic parameters which result in a significant reduction of the number or degree/percentage of subjects with gout flares. The formulations can be modified release dosage forms for once daily administration or immediate release dosage forms administered at least twice daily. In particular, after administration to a subject in need of treatment of a xanthine oxidase inhibitor, the formulations produce in the subject fluctuations in a plasma concentration profile of the xanthine oxidase inhibitor within a certain value for a period after administration to 24 hours, and result in a significant reduction of the number or degree/percentage of subjects with gout flares.

Without being bound by theory, the plasma concentration profile of xanthine oxidase inhibitor which results in a significant reduction of the number or degree/percentage of subjects with gout flares or the degree of gout flares can be characterized by the ratio of maximum plasma concentration (Cₘₐₓ) to minimum plasma concentration profile (Cₘᵢₙ) in the subject for a period of from administration of a single dose to 24 hours. The ratio at steady state can be less than or equal to 80, 70, 60, or 50. In one embodiment, the ratio can be less than or equal to 60. The ratio can be achieved by administration to a subject of an effective amount of a xanthine oxidase inhibitor in a modified release dosage form once daily or in an immediate release dosage form two or more times daily as disclosed herein.

Any of the above methods can further comprise selecting a modified release oral dosage form of the xanthine oxidase inhibitor instead of an immediate release oral dosage form of the xanthine oxidase inhibitor.

The disclosed methods are characterized by the advantage that the number or degree of gout flares experienced by subjects receiving once daily administration of a modified release dosage form or at least twice daily administration of an immediate release dosage form of a xanthine oxidase inhibitor is reduced from the number or degree of gout flares experienced by subjects receiving once daily administration of an immediate release dosage form of the xanthine oxidase inhibitor. In particular, the number or degree of gout flares experienced by subjects receiving once daily administration of a febuxostat modified release dosage form or at least twice daily administration of a febuxostat immediate release dosage form is reduced from the number or degree of gout flares experienced by subjects receiving once daily administration of a febuxostat immediate release dosage form containing 40 mg or 80 mg febuxostat. Also, the number or degree of gout flares experienced by subjects receiving once daily administration of a febuxostat modified release dosage form or at least twice daily administration of a febuxostat immediate release dosage form is reduced from the number or degree of gout flares characterizing once daily administration of an immediate release dosage form of the xanthine oxidase inhibitor, wherein the once daily administration of the modified release dosage form or twice daily administration of the immediate release dosage form shows equivalent or similar serum urate reduction efficacy as the once daily administration of an immediate release dosage form.

The methods are additionally characterized by the advantage that the number or degree of gout flares experienced by subjects receiving once daily administration of a modified release dosage form or at least twice daily administration of an immediate release dosage form of a xanthine oxidase inhibitor is less than or equal to the number or degree of gout flares experienced by subjects receiving administration of placebo. In particular, once daily administration of a febuxostat modified release dosage form is characterized by an incidence of gout flares that is less than or equal to the incidence of gout flares characterizing administration of placebo.

The reduction of incidence or degree of gout flares need not be statistically significant to represent a reduction in gout flares. For example, a clinical trial measuring the incidence or degree of gout flares of a modified release formulation compared to the incidence or degree of gout flares of an immediate release formulation may show a reduction in gout flares lacking statistical significance (a “trend”) in the population studied. A trend is sufficient to establish a modified release formulation reduces the incidence of gout flares compared to an immediate release formulation in, e.g., a larger population.

In any of the methods disclosed herein, administration of the xanthine oxidase inhibitor can be oral administration.

In any of the methods disclosed herein, a prophylactic against gout flares is concomitantly administered to the patient. In some embodiments, the prophylactic is administered concomitantly for the initial period of the ULT with the xanthine oxidase inhibitor. The initial period of administration of the xanthine oxidase inhibitor can be, for example, the first 2 weeks, 1 month, 2 months, 3 months, 4 months, 5 months, 6 months, 9 months, or 12 months after initiation of the ULT with the xanthine oxidase inhibitor. In certain embodiments, the initial period of ULT in which the prophylactic is administered concomitantly with the xanthine oxidase inhibitor is the first six months of initiating administration of the xanthine oxidase inhibitor.

The prophylactic can be colchicine or an NSAID. In some embodiments, the prophylactic is 0.6 mg colchicine administered once daily, or administered every other day for patients with at least moderate renal impairment.

When a prophylactic against gout flares is concomitantly administered to the patient upon initiation of ULT with the xanthine oxidase inhibitor, preventing at least one gout flare or reducing the number or degree of gout flares experienced by the patient occurs during the two month period after cessation of concomitant administration of the prophylactic.

In any of the methods disclosed herein, with concomitant administration of a prophylactic with once daily administration of a modified release dosage form or at least twice daily administration of an immediate release dosage form of the xanthine oxidase inhibitor, the method is characterized by a number or degree of gout flares during the two month period after cessation of concomitant administration of the prophylactic that is less than or equal to the number or degree of gout flares characterizing administration of placebo during that time period.

In any of the methods disclosed herein, the amount of the xanthine oxidase inhibitor in the dosage form can be an effective amount.

Examples of the xanthine oxidase inhibitor for use in any of the methods disclosed herein include febuxostat, topiroxostat, allopurinol, a compound described or claimed in U.S. Pat. No. 7,598,254 (WO2005/121153) or US2012015972 (WO2010/113942 and a triarylcarboxylic acid compound described or claimed in U.S. Pat. No. 7,816,558 (WO2007/043457) or represented by the following formula (I) or a salt thereof, wherein the substituents of formula (I) are as described above.

In certain embodiments of the methods, the xanthine oxidase inhibitor is febuxostat. The febuxostat can be formulated in a modified release dosage form or in an immediate release dosage form. The febuxostat can be present in the dosage form at about 1 mg to about 500 mg, about 1 mg to about 240 mg, about 1 mg to about 120 mg, about 1 mg to about 80 mg, or about 1 mg to about 40 mg. For example, the modified release dosage form or the immediate release dosage form used in the methods can contain about 5 mg, about 30 mg, about 40 mg, or about 80 mg febuxostat. In certain embodiments, an oral modified release dosage form has 40 mg or 80 mg of febuxostat. In certain embodiments, an oral immediate release dosage form has 30 mg of febuxostat. In certain embodiments, an oral immediate release dosage form has 120 mg of febuxostat.

In any of the methods disclosed herein, the patient can have hyperuricemia, gout, acute gouty arthritis, chronic gouty joint disease, tophaceous gout, uric acid nephropathy, or nephrolithiasis. In certain embodiments, the patient has gout with hyperuricemia.

Disclosed herein are methods of preserving renal function of a patient.

In an embodiment, the method comprises administering to a patient with hyperuricemia an effective amount of a xanthine oxidase inhibitor in a modified release dosage form once daily or in an immediate release dosage form two or more times daily to preserve renal function of the patient.

In an embodiment, the method comprises preserving renal function of a patient by administering to a patient with hyperuricemia an effective amount of a xanthine oxidase inhibitor in a modified release dosage form once daily or in an immediate release dosage form two or more times daily.

“Febuxostat therapy” refers to medical treatment of a symptom, disorder, or condition by administration of febuxostat. Febuxostat therapy can be considered optimal when effective plasma levels are reached when required. In addition, peak plasma values (Cₘₐₓ) should be as low as possible so as to reduce the incidence and severity of possible side effects.

A “dosage form” means a unit of administration of an active agent. Examples of dosage forms include tablets, capsules, injections, suspensions, liquids, emulsions, creams, ointments, suppositories, inhalable forms, transdermal forms, and the like. An “oral dosage form” means a unit dosage form for oral administration.

“Dosing regimen” means the dose of an active agent taken at a first time by a patient and the interval (time or symptomatic) at which any subsequent doses of the active agent are taken by the patient. The additional doses of the active agent can be different from the dose taken at the first time.

A “dose” means the measured quantity of an active agent to be taken at one time by a patient.

“Efficacy” means the ability of an active agent administered to a patient to produce a therapeutic effect in the patient.

The term “effective amount” or “therapeutically effective amount” means an amount effective, when administered to a patient, to provide any therapeutic benefit. A therapeutic benefit may be an amelioration of symptoms, e.g., an amount effective to decrease pain. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. An effective amount of an active agent may also be an amount sufficient to provide a significant positive effect on any indicium of a disease, disorder, or condition, e.g. an amount sufficient to significantly reduce the severity of pain. A significant effect on an indicium of a disease, disorder, or condition is statistically significant in a standard parametric test of statistical significance, for example Student's T-test, where p≦0.05. An “effective amount” or “therapeutically effective amount” of febuxostat may be from about 1 mg to about 500 mg, specifically about 5 mg to about 240 mg, more specifically about 10 to about 120 mg febuxostat per day.

The term “equal” means “not significantly different”.

The term “equivalent” means having equal or similar value, meaning, effect, or function.

Two values for a parameter are “similar” when the two values differ by no more than 20%, preferably by no more than 10%.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

“Pharmaceutically acceptable” means that which is generally safe, non-toxic and neither biologically nor otherwise undesirable and includes that which is acceptable for veterinary use as well as human pharmaceutical use.

“Pharmaceutically acceptable salts” includes derivatives of a compound, wherein the compound is modified by making acid or base addition salts thereof, and further refers to pharmaceutically acceptable solvates, including hydrates, and co-crystals of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the compound. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparaginate, glutamate, and the like; and combinations comprising one or more of the foregoing salts. All forms of such derivatives of compound are contemplated herein, including all crystalline, amorphous, and polymorph forms.

“Pharmacokinetic parameters” describe the in vivo characteristics of an active agent (or a metabolite or a surrogate marker for the active agent) over time, such as plasma concentration (C), Cₘₐₓ, Cₙ, C₂₄, Tₘₐₓ, and AUC. “Cₘₐₓ” is the measured plasma concentration of the active agent at the point of maximum, or peak, concentration. “Cₘᵢₙ” is the measured plasma concentration of the active agent at the point of minimum concentration. “Cₙ” is the measured plasma concentration of the active agent at about n hours after administration. “C₂₄” is the measured plasma concentration of the active agent at about 24 hours after administration. The term “Tₘₐₓ” refers to the time at which the measured plasma concentration of the active agent is the highest after administration of the active agent. “AUC” is the area under the curve of a graph of the measured plasma concentration of an active agent vs. time, measured from one time point to another time point. For example AUC₀₋ₜ is the area under the curve of plasma concentration versus time from time 0 to time t, where t can be the last time point with measurable plasma concentration for an individual formulation. The AUC_{0-∞} or AUC_{0-INF} is the calculated area under the curve of plasma concentration versus time from time 0 to time infinity. Similarly, AUC₀₋₄ is the calculated area under the curve of plasma concentration versus time from time 0 to 4 hours and AUC₄₋₂₄ is the calculated area under the curve of plasma concentration versus time from 4 hours to 24 hours after administration. In steady-state studies, AUC_{0-τ} is the area under the curve of plasma concentration over the dosing interval (i.e., from time 0 to time τ (tau), where tau is the length of the dosing interval. Other pharmacokinetic parameters are the parameter Kₑ or Kₑₗ, the terminal elimination rate constant calculated from a semi-log plot of the plasma concentration versus time curve; t_{1/2} the terminal elimination half-life, calculated as 0.693/Kₑₗ; CL/F denotes the apparent total body clearance after administration, calculated as Total Dose/Total AUC; and Vₐᵣₑₐ/F denotes the apparent total volume of distribution after administration, calculated as Total Dose/(Total AUC_{∞}×Kₑₗ).

Mean Residence Time (“MRT”) is the average time a drug spends in a compartment or system, and is equal to AUMC/AUC. MRTinf is the mean residence time extrapolated to infinity and equals AUMCinf/AUCinf. AUMC is the area under the moment curve, and AUMCinf is the area under the moment curve extrapolated to infinity. AUMCinf is calculated with the following equation:

$$A U M Cinf = \frac{A U M C {\int }_{0}^{τ} + τ ( A U C inf - A U C {\int }_{0}^{τ} )}{A U C {\int }_{0}^{τ}} ,$$

“Side effect” means a secondary effect resulting from taking an active agent. The secondary effect can be a negative (unfavorable) effect (i.e., an adverse side effect) or a positive (favorable) effect.

The term “subject” includes any human or non-human animal. For example, the methods and compositions disclosed herein can be used to treat a subject having hyperuricemia. In a particular embodiment, the subject is a human.

The terms “treating” and “treatment” mean implementation of therapy with the intention of reducing in severity or frequency symptoms, elimination of symptoms or underlying cause, prevention of the occurrence of symptoms or their underlying cause, and improvement or remediation of damage.

The terms “administer”, “administering”, “administered” or “administration” refer to any manner of providing an active agent (such as, febuxostat or a pharmaceutically acceptable salt thereof) to a subject or patient. Routes of administration can be accomplished through any means known by those skilled in the art. Such means include oral, buccal, intravenous, subcutaneous, intramuscular, transdermal, and inhalation.

The team “immediate-release” refers to a pharmaceutical formulation characterized by conventional or non-modified release of the active agent immediately after drug administration. In some embodiments, immediate release means greater than or equal to about 75% of the active agent is released within two hours of administration, specifically within one hour of administration.

As used herein, the term “modified release” refers to a pharmaceutical formulation in which release of the active agent is not immediate (See, for example, Guidance for Industry SUPAC-MR: Modified Release Solid Oral Dosage Forms, Scale-Up and Postapproval Changes: Chemistry, Manufacturing, and Controls; In Vitro Dissolution, Testing and In Vivo Bioequivalence Documentation, U.S. Department of Health and Human Services, Food and Drug Administration, Center for Drug Evaluation and Research (“CDER”), September 1997 CMC 8, page 34, herein incorporated by reference.). The term is used interchangeably with “nonimmediate release” as defined in Remington: The Science and Practice of Pharmacy, Nineteenth Ed. (Easton, Pa.: Mack Publishing Company, 1995). As used herein, the term “modified release” includes extended or controlled release, delayed release, and delayed-controlled release formulations.

As used herein, the term “extended release” refers to a pharmaceutical formulation that provides for the gradual release of an active agent over an extended period of time. “Extended-release” includes the release of the active agent at such a rate that blood (e.g., plasma) levels are maintained within a therapeutic range for at least about 5 hours, specifically at least about 12 hours, and more specifically at least about 24 hours after administration at steady-state. The term steady-state means that a plasma level for a given active agent has been achieved and which is maintained with subsequent doses of the drug at a level which is at or above the minimum effective therapeutic level for a given active agent.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption occurs in the small intestine.

An extended-release form is a form suitable for providing controlled-release of febuxostat over a sustained period of time (e.g., 5 hours, 12 hours, 24 hours). Extended-release dosage forms of febuxostat may release the active agent at a rate independent of pH, for example, about pH 1.2 to about 7.5. Alternatively, extended-release dosage forms may release febuxostat at a rate dependent upon pH, for example, a lower rate of release at pH 1.2 and a higher rate of release at pH 6.8. Specifically, the extended-release form avoids dose dumping upon oral administration. The extended-release oral dosage form can be formulated to provide for an increased duration of febuxostat action allowing once-daily dosing.

The term “controlled” release refers to a type of extended release formulation in which the gradual release of the active agent is controlled or manipulated over a certain extended period of time.

Active agent release from a pharmaceutical formulation can be analyzed in various ways. One exemplary test is in vitro dissolution. A dissolution profile is a plot of the cumulative amount of active agent released from a formulation as a function of time. A dissolution profile can be measured utilizing the Drug Release Test <724>, which incorporates standard test USP 28 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

For example, for febuxostat dosage forms, febuxostat release characteristics and dissolution profiles, can be evaluated in 900 mL of 0.5 M phosphate buffer, pH 6.8, equilibrated at 37° C.±0.5° C. using a paddle method (USP Apparatus 2) at 50 rpm. Other conditions, such as different pH, may be used as known in the art. Sample aliquots can be taken at different time intervals and analyzed by high performance liquid chromatography.

Alternatively, active agent release from a pharmaceutical formulation can be determined in a pharmacokinetics study. Design of such a pharmacokinetics study is within the skill of practitioners in the art.

The modified release febuxostat dosage forms when orally administered once daily to a subject, provide a high percentage of xanthine oxidase inhibition while producing a maximum observed plasma concentration (Cₘₐₓ) that is lower than that provided by an immediate release febuxostat dosage form containing about 5 mg, about 10 mg, about 20 mg, about 40 mg, about 80 mg, about 120 mg, or about 240 mg of febuxostat administered to a subject once daily.

In an embodiment, oral administration to a subject of a modified release febuxostat dosage forms should maintain in the subject, a plasma concentration of febuxostat or a pharmaceutically acceptable salt thereof greater than about 0.05 μg/mL to about 0.1 μg/mL for a period of from about 5 to about 24 hours. More specifically, oral administration of the modified release febuxostat dosage forms can maintain in the subject, a plasma concentration of febuxostat or pharmaceutically acceptable salt thereof greater than about 0.1 μg/mL for a period for about 4.0 hours, about 5.0 hours, for about 6.0 hours, for about 7.0 hours, for about 8.0 hours, for about 9.0 hours, for about 10.0 hours, for about 11.0 hours, for about 12.0 hours, for about 13.0 hours, for about 14.0 hours, for about 15.0 hours, for about 16.0 hours, for about 17.0 hours, for about 18.0 hours, for about 19.0 hours, for about 20.0 hours, for about 21.0 hours, for about 22.0 hours, for about 23.0 hours or for about 24.0 hours

In an embodiment, the modified release dosage form provides, after administration of a single dose, a mean residence time (MRTinf) of the febuxostat of at least 7 hours. In an embodiment, the MRTinf is at least 8 hours, at least 9 hours, at least 10 hours, at least 11 hours, or at least 12 hours. In an embodiment, the MRTinf has a value between about 7 hours and about 16 hours, about 8 hours and about 15 hours, about 9 hours and about 14 hours, about 10 hours and about 13 hours, or about 11 hours and about 13 hours. In an embodiment, the MRTinf about 12 hours.

In an embodiment, the modified release dosage form provides, after administration of a single dose, a Cmax per dose strength of less than about 20 ng/mL/mg. In some embodiments, the Cmax per dose strength is less than about 19 ng/mL/mg, less than about 18 ng/mL/mg, less than about 17 ng/mL/mg, less than about 16 ng/mL/mg, less than about 15 ng/mL/mg, less than about 14 ng/mL/mg, or less than about 13 ng/mL/mg. In an embodiment, the Cmax per dose strength is between about 11 ng/mL/mg to about 13 ng/mL/mg.

In an embodiment, the modified release dosage form provides, after administration of a single dose, a Cmax of less than about 1500 ng/mL. In an embodiment, Cmax is less than about 1400 ng/mL, less than about 1200 ng/mL, less than about 1100 ng/mL, or less than about 1000 ng/mL. In an embodiment, Cmx is in the range of about to about 900 ng/ml to about 1500 ng/ml. In an embodiment, the Cmax is in the range of about 950 ng/ml to about 1450 ng/ml, or about 980 ng/ml to about 1400 ng/ml.

In an embodiment, the modified release dosage form provides, after administration of a single dose, a Cmax of less than about 750 ng/mL. In an embodiment, Cmax is less than about 700 ng/mL, less than about 600 ng/mL, less than about 550 ng/mL, or less than about 500 ng/mL. In an embodiment, Cmax is in the range of about to about 450 ng/ml to about 750 ng/ml. In an embodiment, the Cmax is in the range of about 475 ng/ml to about 725 ng/ml, or about 490 ng/ml to about 700 ng/ml.

In an embodiment, the modified release dosage form provides, after administration of a single dose, a Tmax in the range of about 2 hours to about 8 hours. In an embodiment, Tmax is in the range of about 3 hours to about 7 hours, about 4 hours to about 7 hours, about 5 hours to about 7 hours. In an embodiment, Tmax is about 6 hours.

In an embodiment, the modified release dosage form provides, after administration of a single dose, an area under the curve from time 0 to 4 hours (AUC₀₋₄) of less than about 1800 hr-ng/mL. In an embodiment, AUC₀₋₄ is less than about 1800 hr-ng/mL, about 1600 hr-ng/mL, about 1400 hr-ng/mL, about 1200 hr-ng/mL, or about 1000 hr-ng/mL. In an embodiment, AUC₀₋₄ is in a range of about 800 hr-ng/mL to about 2000 hr-ng/mL. In an embodiment, AUC₀₋₄ is in a range of about 850 hr-ng/mL to about 1800 hr-ng/mL, about 900 hr-ng/mL to about 1600 hr-ng/ml, about 900 hr-ng/mL to about 1400 hr-ng/ml, about 900 hr-ng/mL to about 1200 hr-ng/ml.

In an embodiment, the modified release dosage form provides, after administration of a single dose, an area under the curve from time 4 hours to time 24 hours (AUC₄₋₂₄) is more than about 4000 hr-ng/mL. In an embodiment, AUC₄₋₂₄ is more than about 4100 hr-ng/mL, about 4200 hr-ng/mL, about 4300 hr-ng/mL, about 4400 hr-ng/mL, about 4500 hr-ng/mL, about 4500 hr-ng/mL, or about 4700 hr-ng/mL. In an embodiment, AUC₄₋₂₄ is in a range of about 4000 hr-ng/mL to about 5000 hr-ng/mL, about 4200 hr-ng/mL to about 4900 hr-ng/mL, about 4400 hr-ng/mL to about 4900 hr-ng/mL, or about 4600 hr-ng/mL to about 4900 hr-ng/mL.

In any of the above embodiments of a febuxostat modified release dosage form, the dosage strength is about 40 mg or 80 mg. In any of the above embodiments of a febuxostat modified release dosage form, the effective amount is about 80 mg.

In an embodiment, the method of reducing the number or degree of gout flares is a method of treating a hyperuricemic patient and reducing the risk the patient experiences a gout flare.

Oral administration to a subject of a xanthine oxidoreductase inhibitor dosage form should produce in the subject fluctuations in the plasma concentration profile of the xanthine oxidoreductase inhibitor within a certain value for a period after administration to 24 hours at steady state. More specifically, oral administration to a subject of a xanthine oxidoreductase inhibitor dosage form should produce in the subject a ratio of the maximum plasma concentration (Cₘₐₓ) to the minimum plasma concentration profile (Cₘᵢₙ) of the xanthine oxidoreductase inhibitor less than or equal to 80, 70, 60 or 50 for a period of from administration to 24 hours at steady state. In particular, oral administration to a subject of the xanthine oxidoreductase inhibitor dosage form should produce in the subject a Cₘₐₓ/Cₘᵢₙ ratio of the xanthine oxidoreductase inhibitor less than or equal to 60 or 50 for a period of from administration to 24 hours at steady state.

The benefits of the present disclosure are not limited to a single type of dosage form and/or dosing regimen.

One embodiment of such dosage forms of febuxostat in combination with dosing regimens is at least twice daily administration of formulations of immediate release dosage forms which are disclosed in WO2003/082279 (US20050043375), incorporated by reference herein.

Another embodiment is once daily administration of modified release dosage forms having specific in vitro release characteristics.

Such modified release dosage forms are formulations having an in vitro dissolution profile of the xanthine oxidoreductase inhibitor of:
(A): a) 20-60% released after 30 min; b) 70-100% released after 60 min; wherein % released is relative to the total amount of the xanthine oxidoreductase inhibitor in the dosage form, measured using USP Apparatus 1 in 900 mL of 50 mM phosphate buffer at pH 6.90 with stirring at 100 rpm at 37° C. in the case of a modified release dosage form comprising a membrane controlled system, more specifically in the case of a modified release dosage form comprising a combination of an immediate release form and a delayed release form comprising a membrane controlled system using an enteric coating to control release of the xanthine oxidase inhibitor;
(B): a) 30-60% released after 60 min; b) 45-75% released after 120 min; c) 70-100% released after 240 min; wherein % released is relative to the total amount of the xanthine oxidoreductase inhibitor in the dosage form, measured using USP Apparatus 1 in 900 mL of 50 mM phosphate buffer at pH 7.20 with stirring at 100 rpm at 37° C. in the case of a modified release dosage form comprising a membrane controlled system, more specifically in the case of a modified release dosage form comprising a combination of an immediate release form and a delayed release form comprising a membrane controlled system using a pH independent polymer coating to control release of the xanthine oxidase inhibitor; or
C: a) 25-55% released after 120-240 min; b) 80-100% released after 180-330 min; wherein % released is relative to the total amount of the xanthine oxidoreductase inhibitor in the dosage form, measured by a dissolution test using the modified paddle method of the dissolution test of the Japanese Pharmacopoeia with a stationary basket operated at pH 6.0, 37° C. with stirring at 200 rpm in the case of matrix system as one of the modified release dosage forms, more specifically in the case of matrix system with immediate release core as the matrix system; or
D; a) 25-55% released after 120-240 min; b) 50-70% released after 180-330 min; wherein % released is relative to the total amount of the xanthine oxidoreductase inhibitor in the dosage form measured by a dissolution test using the modified paddle method of the dissolution test of the Japanese Pharmacopoeia with a stationary basket operated at pH6.0, 37° C. with stirring at 200 rpm in the case of a modified release dosage form comprising a matrix system, more specifically in the case of a modified release dosage form comprising a matrix system comprising a sustained release core.

Such modified release dosage forms are not limited to a single type of dosage form having a particular mechanism of drug release. These desired dissolution profiles can be obtained with any system of oral modified release dosage form known in the art. Three different examples of oral modified release dosage forms, namely, matrix systems, osmotic pumps, and membrane controlled technology, are described in greater detail below. However, although these three oral modified release dosage forms are described in greater detail, other modified release dosage forms known to those skilled in the art can be used. A detailed discussion of various modified release dosage forms may be found in: (i) Handbook of pharmaceutical controlled release technology, ed. D. L. Wise, Marcel Dekker, Inc. New York, N.Y. (2000), and (ii). Treatise on controlled drug delivery, fundamentals, optimization, and applications, ed. A. Kydonieus, Marcel Dekker, Inc. New York, N.Y. (1992), the contents of each which is hereby incorporated by reference.

Membrane controlled systems are well known in the art. This technology is also commonly referred to as a reservoir system, microencapsulation, bead technology, or coated tablets. Particles or tablets containing the drug are encapsulated or coated with pharmaceutically acceptable polymer(s) such as enteric coating polymer or pH independent polymer. This polymer, and its relative quantity, offers a predetermined resistance to drug diffusion from the reservoir to the gastrointestinal tract. Thus, the drug is gradually released from the beads or tablet into the gastrointestinal tract and provides the desired controlled release of the drug. These dosage forms are well known in the art. For example, U.S. Pat. Nos. 5,286,497 and 5,737,320 and U.S. Patent Application No. 2011311620 describe such formulations and their methods of production. One skilled in the art, taking into account the teachings in this application as well as those of U.S. Pat. Nos. 5,286,497 and 5,737,320 and U.S. Patent Application No. 2011311620, could produce a tablet, bead, or pellet-based dosage form matching a pharmacokinetic and/or a dissolution profile described above.

Matrix systems are well known in the art. In a matrix system, the drug is admixed with a polymer, optionally in association with additional conventional excipients. This admixture is typically compressed under pressure to produce a tablet. Drug is released from this tablet by diffusion and erosion. Matrix systems are described in detail by either Wise or Kydonieus, supra. A modified release dosage form comprising a matrix system can contain a matrix system-controlled release outer coating on a core. This type of modified release dosage form is described in U.S. Patent Application No. 2013/0089609.

Osmotic pump systems are well known in the art and have been described in the literature. U.S. Pat. Nos. 4,088,864; 4,200,098; 5,573,776; and U.S. Patent Application 2011311620, all of which are hereby incorporated by reference, describe osmotic pumps and methods for their manufacture. In an osmotic pump system, a tablet core is encased by a semipermeable membrane having at least one orifice. The semipermeable membrane is permeable to water, but impermeable to the drug. When the system is exposed to body fluids, water will penetrate through the semipermeable membrane into the tablet core containing osmotic excipients and the active drug. Osmotic pressure increases within the dosage form and drug is released through the orifice in an attempt to equalize pressure.

Examples of such modified release febuxostat dosage forms meeting one or more of these above characteristics are disclosed in U.S. Patent Application 2011311620 (membrane controlled system, matrix system, and osmotic pump system) and U.S. Patent Application 20130089609 (matrix system), incorporated by reference herein. A modified release febuxostat dosage form can contain, for example, about 5 mg, about 10 mg, about 20 mg, about 30 mg, about 40 mg, about 80 mg, or about 120 mg febuxostat.

In an embodiment, the febuxostat modified release dosage form comprises about 10% to about 30%, specifically about 20%, of the febuxostat in an immediate release form and about 90% to about 70%, specifically about 80%, of the febuxostat in a delayed release form, wherein the % febuxostat is based on the total amount of febuxostat in the modified release dosage form. The febuxostat modified release dosage form can be in the form of an oral capsule or tablet containing two types of febuxostat beads. One type of bead can be an immediate release febuxostat bead. In an embodiment, the immediate release febuxostat bead comprises febuxostat layered on an inert core, such as sugar spheres or microcrystalline cellulose spheres, by means of a suitable polymeric binder. The polymeric binder can be hydroxypropyl methylcellulose. The additional type of bead can be a delayed release bead. The delayed release beads can be coated beads obtained by coating immediate release beads with a delayed release enteric polymer either in an aqueous dispersion or in an organic solvent. These polymers can have pH dependent solubility depending on the functional groups on the polymer. For a delayed release bead coated with suitable amount of delayed release enteric polymer, drug release will not occur in a medium unless medium pH is above the pH at which the polymer dissolves. The delayed release enteric polymers of the delayed release febuxostat bead become soluble when the bead is exposed to a pH level generally less acidic than the environment of the stomach. Specifically, the delayed release polymer may become soluble at pH levels greater than or equal to 4.5; 4.6; 4.7; 4.8; 4.9; 5.0; 5.1; 5.2; 5.3; 5.4; 5.5; 5.6; 5.7; 5.8; 5.9; 6.0; 6.1; 6.2; 6.3; 6.4; 6.5; 6.6; 6.7; 6.8; 6.9; 7.0; 7.1; 7.2; 7.3; 7.4; 7.5; 7.6; 7.7; 7.8; 7.9; 8.0; 8.1; 8.2; 8.3; 8.4; 8.5; 8.6; 8.7; 8.8; 8.9; 9.0; 9.1; 9.2; 9.3; 9.4; 9.5; 9.6; 9.7; 9.8; 9.9; or 10.0. In an embodiment, the delayed release polymer becomes soluble at pH levels greater than or equal to 5.5, 6.0, or 6.8, specifically at pH ≧6.8. In an embodiment, the delayed release polymer can be a methacrylic acid copolymer, or a combination of methacrylic acid copolymers, providing the desired pH release.

In some embodiments of any of the above methods, the patient can have renal impairment. Approximately 40% to 60% of patients with hyperuricemia and gout have some degree of renal impairment. In certain embodiments of the methods, the patient can have mild renal impairment, moderate renal impairment, severe renal impairment, or ends stage renal disease. One measure of renal impairment is estimated glomerular filtration rate (eGFR). Herein, mild renal impairment corresponds to a value of eGFR of 60-89 mL/min, moderate renal impairment corresponds to a value of eGFR ≧30 and ≦59 mL/min, specifically eGFR ≧30 and ≦50 mL/min, and severe renal impairment corresponds to a value of eGFR ≧15 and <30 mL/min. In certain embodiments the patient can have end stage renal disease (eGFR value <15 mL/min. Normal renal function corresponds to eGFR ≧90 mL/min.

Pharmaceutical compositions for hyperuricemia containing a xanthine oxidase inhibitor for preventing gout flares or reducing the number or degree of gout flares associated with urate-lowering therapy are also disclosed. Pharmaceutical compositions for hyperuricemia containing a xanthine oxidase inhibitor in a modified release dosage form with no need of administration in a dose-escalating (i.e. dose titration) regimen are also disclosed. The xanthine oxidase inhibitor can be febuxostat, topiroxostat, allopurinol, a compound described or claimed in U.S. Pat. No. 7,598,254 (WO2005/121153) or US2012015972 (WO2010/113942), or a triarylcarboxylic acid compound described or claimed in U.S. Pat. No. 7,816,558 (WO2007/043457) or represented by the following formula (I) or a salt thereof:

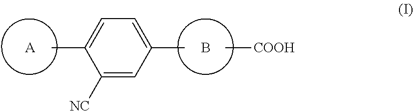

wherein: A: aryl or heteroaryl, wherein aryl and heteroaryl may be substituted with the same or different, 1 to 3 substituents selected from the following group G;

group G: halogen, —CN, —NO2, lower alkyl, halogeno-lower alkyl, —O—R1, —O-halogeno-lower alkyl, —O—CO—R1, —O-benzyl, —O-phenyl, —NR2R3, —NH—CO—R1, —CO2-R1, —CO—R1, —CO—NR2R3, —CO-phenyl, —S—R1, —SO2-lower alkyl, —SO2-phenyl, —NH—SO2-naphthalene-NR2R3, phenyl, cycloa&yl, and -lower alkylene-O—R1;

R1: H or lower alkyl;

R2 and R3: same or different, each representing H or lower alkyl,

wherein R2 and R3, taken together with the nitrogen atom to which they bond, may form a monocyclic nitrogen-containing saturated heterocycle; and

B: monocyclic heteroaryl, wherein the monocyclic heteroaryl may be substituted with a group selected from lower alkyl, —OH, and halogen.

In an embodiment, the pharmaceutical composition is a modified release dosage form that is administered once daily. In certain embodiments, the xanthine oxidase inhibitor is febuxostat and the amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg.

In an embodiment, the pharmaceutical composition is an immediate release xanthine oxidase inhibitor dosage form that is administered at least twice daily. In certain embodiments, the xanthine oxidase inhibitor is febuxostat and the amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, or about 1 mg to about 40 mg.

In an embodiment, the pharmaceutical composition is a modified release febuxostat dosage form that is administered once daily, wherein the amount of febuxostat in the modified release dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein the number or degree of gout flares characterizing the once daily administration of the febuxostat modified release dosage form is reduced from the number or degree of gout flares characterizing once daily administration of a febuxostat immediate release dosage form containing 40 mg or 80 mg febuxostat.

In an embodiment, the pharmaceutical composition is a modified release febuxostat dosage form that is administered once daily, wherein the amount of febuxostat in the modified release dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein the number or degree of gout flares characterizing the once daily administration of the febuxostat modified release dosage form is reduced from the number or degree of gout flares characterizing once daily administration of an immediate release dosage form of the xanthine oxidase inhibitor, wherein the once daily administration of the modified release dosage form or twice daily administration of the immediate release dosage form shows equivalent or similar serum urate reduction efficacy as the once daily administration of an immediate release dosage form.

In an embodiment, the pharmaceutical composition is a modified release febuxostat dosage form administered once daily, wherein the amount of febuxostat in the modified release dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein once daily administration of the febuxostat modified release dosage form is characterized by the number or degree of gout flares that is less than or equal to the number or degree of gout flares characterizing administration of placebo.

In an embodiment, the pharmaceutical composition is an immediate release febuxostat dosage form that is administered at least twice daily, wherein the amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein the number or degree of gout flares characterizing the at least twice daily administration of the febuxostat immediate release dosage form is reduced from the number or degree of gout flares characterizing once daily administration of a febuxostat immediate release dosage form containing 40 mg or 80 mg febuxostat.

In an embodiment, the pharmaceutical composition is an immediate release febuxostat dosage form that is administered at least twice daily, wherein the amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein the number or degree of gout flares characterizing once daily administration of an immediate release dosage form of the xanthine oxidase inhibitor, wherein the once daily administration of the modified release dosage form or twice daily administration of the immediate release dosage form shows equivalent or similar serum urate reduction efficacy as the once daily administration of an immediate release dosage form.

In an embodiment, the pharmaceutical composition is an immediate release febuxostat dosage form administered at least twice daily, wherein the amount of febuxostat in the dosage form can be about 1 mg to about 500 mg, about 1 mg to about 240 mg, about 1 mg to about 120 mg, about 5 mg to about 120 mg, about 1 mg to about 80 mg, about 5 mg to about 80 mg, about 10 mg to about 50 mg, about 1 mg to about 40 mg, wherein at least twice daily administration of the febuxostat immediate release dosage form is characterized by a number of gout flares that is less than or equal to the number or degree of gout flares characterizing administration of placebo.

In any of the above agents, the pharmaceutical composition can be administered to a patient who is in need of initiating urate-lowering therapy. For example, the patient can have hyperuricemia, gout, acute gouty arthritis, chronic gouty joint disease, tophaceous gout, uric acid nephropathy, or nephrolithiasis. In an embodiment, the patient has gout with hyperuricemia.

The following examples further illustrate the invention but should not be construed as in any way limiting its scope.

### EXAMPLES

### Example 1

A multicenter, randomized, double-blind study was designed and performed to evaluate the effect of febuxostat compared to placebo on renal function in hyperuricemic (sUA >7.0 mg/dL) gout subjects.

The primary objective of this 12 month study was to evaluate the effect of treatment with febuxostat 40 mg/80 mg IR QD and febuxostat 30 mg IR BID on renal function compared with placebo in hyperuricemic gout subjects with moderate to severe renal impairment.

The secondary objective of this study was to evaluate the pharmacokinetics and pharmacodynamics of febuxostat in hyperuricemic gout subjects with moderate to severe renal impairment.

All subjects met the American Rheumatism Association (ARA) diagnostic criteria for gout with the exception of the criteria related to tophi. Subjects with tophaceous gout at screening were excluded. Up to 90 subjects were planned to enroll at approximately 75 US sites.

Subjects who met the enrollment criteria were randomized to 1 of 3 arms in a 1:1:1 ratio to receive either daily febuxostat 40 mg/80 mg QD, febuxostat 30 mg BID, or placebo for up to 12 months. The overall duration of the study was approximately 14 months (12 months of active drug treatment). Randomization was stratified at baseline using 3 strata: subjects taking an angiotensin receptor blocker (ARB), subjects taking an angiotensin converting enzyme inhibitors (ACEi), or subjects not taking an ARB or an ACEi.

Subjects were screened at Day −21 for entry.

All subjects received colchicine 0.6 mg every other day (QOD) from the initial screening visit through the Month 6 visit only. Alternatively, if colchicine was not tolerated by the subject, prednisone may have been provided at the investigator's discretion in accordance with the stated guidelines listed under Prohibited Concomitant Medications. Nonsteroidal anti-inflammatory drugs (NSAIDs) were not permitted during the study. Gout flares were also treated throughout the study at the discretion of the investigator, and in compliance with the protocol.

Subjects randomized to the QD febuxostat group initially received 40 mg QD and remained on 40 mg QD for the remainder of the study if their sUA was <6.0 mg/dL at the Day 14 visit. Subjects whose sUA was ≧6.0 mg/dL at the Day 14 visit received febuxostat 80 mg QD at the Month 1 visit, and remained on this dose for the remainder of the study. Subjects randomized to the placebo or febuxostat 30 mg BID groups did not have their treatment changed during the study.

Following the Study Day 1 Visit, subjects returned to the clinic for study visits at Day 14, Months 1, 3, 6, 9 and for a fmal visit at Month 12/ET Visit. Blood samples were collected at various visits for the analysis of febuxostat concentrations to evaluate the PK of febuxostat. Gout flare assessments were collected at each visit. Estimated GFR by Modification of Diet in Renal Disease (MDRD) calculation was performed by the Central Laboratory at all visits. Blood pressure (clinic with standardized instruments) was measured throughout the study. Adverse events, electrocardiograms (ECGs), clinical laboratory tests, and vital signs were collected at each visit.

Subjects maintained their usual and customary fluid and dietary patterns throughout the study. However, subjects were instructed to fast for clinical laboratory tests at least 8 hours prior to returning to the investigative site at Randomization (Day 1) and Month 12/ET visit. If a subject did not fast prior to a scheduled fasting lab visit, an unscheduled visit was not required to obtain the fasting labs. Subjects were not required to fast at any of the Screening Visits (or prior to signing informed consent), Months 1, 3, 6, and 9.

Subjects who received colchicine for gout flare prophylaxis were to avoid eating grapefruit and Seville oranges, or drinking grapefruit juice or Seville orange juice.

Subjects completed the patient reported outcomes questionnaire, the Short Form version 2 (SF-36v2) at Day 1, Months 6, and 12/ET visits. Subjects were contacted by telephone prior to Months 3, 6, 9 and 12/ET visits to confirm dosing times of the study medication prior to the scheduled visits.

Efficacy and safety were assessed throughout the study. The schedule for all study-related procedures for all evaluations is shown in Table 1. Assessments were completed at the designated visit/time point(s). Study days/weeks and visit windows were calculated after randomization and were calculated from the day of the first dose of double-blind treatment (Day 1). The length of study participation for each subject was expected to be approximately 14 months.

TABLE 1
Schedule of Study Assessments and Procedures

| | Study Day: | | | | | | | | | | |
|---|---|---|---|---|---|---|---|---|---|---|---|
| | | | | | | | | | | | Month 12/ |
| | | | | | | | | | | | Early |
| | Screening | | | | | | | | | | Termination |
| | Day −21 | | Day −14 | Day −7 | Day 1 | Day 14 | Month 1 | Month 3 | Month 6 | Month 9 | Visit (ET) |
| | Visit Windows: | | | | | | | | | | |
| | | | (+3 days) | (+3 days) | | (+4 days) | (±4 days) | (±4 days) | (±4 days) | (±4 days) | (±4 days) |
| | Visit Numbers: | | | | | | | | | | |
| | 1 | 2(b) | 3 | 4 | 5 | 6 | 7 | 8 | 9 | 10 | 11 |
| Obtained informed | X | | | | | | | | | | |
| consent | | | | | | | | | | | |
| Call IVRS | X | | | | X | | X | X | X | X | X |
| Dispense gout flare | X (a) | | | | | | | X | | | |
| prophylactic | | | | | | | | | | | |
| medication | | | | | | | | | | | |
| Dispense study | | | | | X | | X (c) | X | X | X | |
| medication | | | | | | | | | | | |
| Inclusion/exclusion | X | | X | X | X | | | | | | |
| criteria | | | | | | | | | | | |
| Prior medication | X | | | | | | | | | | |
| assessment | | | | | | | | | | | |
| Demographics, | X | | | | | | | | | | |
| medical and | | | | | | | | | | | |
| social history, | | | | | | | | | | | |
| Height and Weight | X (d) | | | | | | | | | | X |
| Physical examination | X | | | | X | | | | X | | X |
| Concurrent | X | | | | | | | | | | |
| medical | | | | | | | | | | | |
| conditions | | | | | | | | | | | |
| Randomization | | | | | X | | | | | | |
| to treatment | | | | | | | | | | | |
| Vital signs (e) | X | | X | X | X (f) | X | X | X | X | X | X |
| 12-lead Safety | | | | | X | | | | | | X |
| ECG | | | | | | | | | | | |
| Clinical | X | | X | X | X (h) | X | X | X | X | X | X (g) |
| laboratory | | | | | | | | | | | |
| tests - 18 (g) | | | | | | | | | | | |
| Serum FSH (i) | X | | | | | | | | | | |
| (sUA (j) | X (k) | | | | X | X | X | X | X | X | X |
| Serum Creatinine | X | | X | X | X | X | X | X | X | X | X |
| and Estimated | | | | | | | | | | | |
| Creatinine | | | | | | | | | | | |
| clearance (k) | | | | | | | | | | | |
| Population PK | | | | | | | | X (l, m) | X (l, m) | X (l, m) | X (l) |
| samples | | | | | | | | | | | |
| SF-36v2 (n) | | | | | X | | | | X | | X |
| Concomitant | X | | X | X | X | X | X | X | X | X | X |
| Medication | | | | | | | | | | | |
| assessment | | | | | | | | | | | |
| Adverse event | | | X | X | X | X | X | X | X | X | X |
| assessment (o) | | | | | | | | | | | |
| Pretreatment event | X | | | | | | | | | | |
| assessment (o) | | | | | | | | | | | |
| Educate subject on | X | | X | X | X | X | X | X | X | X | X |
| gout flare | | | | | | | | | | | |
| reporting (p) | | | | | | | | | | | |
| Tophi assessment | | | | | | X | X | X | X | X | X |
| (if present) (q) | | | | | | | | | | | |
| Collect unused | | | | | | | | X | X | | |
| prophylactic | | | | | | | | | | | |
| medication | | | | | | | | | | | |
| Collect unused | | | | | | | X | X | X | X | X |
| study medication | | | | | | | | | | | |
| Telephone contact | | | | | | | | X | X | X | X |
| to subjects (r) | | | | | | | | | | | |

(a) Prophylaxis colchicine was dispensed at the Screening Day −21 visit for all subjects.
(b)Visit removed.
(c) Subjects remained on 40 mg febuxostat for the remainder of the study if their sUA was <6.0 mg/dL at the Day 14 visit. Subjects whose sUA was ≧6.0 mg/dL at the Day 14 visit received febuxostat 80 mg QD at Month 1 through the remainder of the study. Subjects randomized to placebo or febuxostat 30 mg BID did not have their treatment changed during the study.
(d) Height was done only at Screening Day −21.
(e) Site collected 3 sitting BP readings and recorded all readings and the average on the eCRF.
(f) Subject was to have an average sitting BP measurements below 160 mmHg systolic and below 95 mmHg diastolic on Day 1 visit plus at least 1 of the prior screening visits. The average sitting BP measurement was used to determine eligibility.
(g) Laboratory Assessment: hematology, urinalysis, chemistry. Prothrombin time, activated partial thromboplastin time (aPTT) and international normalized ratio (INR) (for subjects on warfarin). sUA and lipid profile were measured as part of the chemistry panel.
(h) Fasting lipid panel was collected at Day 1 and Month 12/ET visits only; fasting occurred 8 hours prior to returning to the site.
(i) Female subjects had an FSH ≧40 IU/L to confirm their post menopausal status at the initial screening visit only; OR female receiving HRT was ≧55 years of age (FSH level not required).
(j) sUA was measured as part of the serum chemistry panel and was blinded to the Sponsor and investigative site beginning at Day 1 through the Month 12/ET visits. A member of the TGRD Pharmacovigilance Department not involved with the febuxostat program was contacted by the central laboratory in the event of a sUA ≦2 mg/dL or >18 mg/dL at any visit. The same member followed up with the study site.
(k) Subjects who do not meet the sUA inclusion criteria (sUA >7.0 mg/dL), serum creatinine ≧1.5 mg/dL and eGFR ≧15-≦50 mL/min at the Day −21 screening visit were withdrawn from the study.
(l) Trough blood samples (2) for PK analyses were collected prior to dosing (−0.25 to 0 hr) at any of the 2 following study visits: Months 3, 6, 9 and/or 12/ET visit.
(m) Nontrough blood samples (4) for PK analyses were collected after dosing and may have been split between study visits. PK samples were collected over the following visits days and intervals: Months 3, 6, and/or 9: 0.25 hr; 0.75 to 2.0 hr; 2.5 to 4.0 hr; and 5 to 12 hr post dose.
(n) SF-36v2 was collected under the supervision of the study coordinator or physician prior to any interaction with the site personnel prior to any clinical assessments.
(o) Adverse events of rash were collected from the time of informed consent and until 30 days after study medication was discontinued. Only spontaneous reported AEs were collected within 30 days after the last dose of study medication. AEs of rash were recorded on the Rash AE Worksheet (Protocol Amendment 3 Appendix G) and transcribed over to the AE source document and eCRF. Rash AE Worksheet were to be faxed to the Sponsor within 48 hours of site knowledge of the event.
(p) Gout flare assessment information reported by the subject were collected by site personnel onto the Gout Flare Assessment Worksheet and later transcribed to the eCRF. In addition, if the subject did not have a reported gout flare during a visit, the site asked the subject 2 additional exploratory questions, which were captured on the Gout Flare Assessment Worksheet.
(q) If tophi developed after randomization, assessment for the presence or absence continued at subsequent visits. Tophi are part of gout and were not captured as an AE.
(r) The investigator site staff contacted the subject prior to the scheduled clinic visit to remind the subject to document the exact times the morning and evening doses of study medication were taken and which were recorded on the subject's source documents and eCRF.

Efficacy of febuxostat was assessed by measurement of serum creatinine (and calculation of eGFR), clinic systolic and diastolic BP, and sUA levels.

Serum samples for the analysis of serum creatinine (and calculation of eGFR) were collected at the time points stipulated in Schedule of Study Procedures (Table 1.). All samples were collected in accordance with standard laboratory procedures. Analyses were conducted by the Central Laboratory as part of the standard clinical laboratory tests.

At each visit, clinic BP measurements were measured while subjects were in a sitting position after they have been seated for at least 5 minutes and in accordance with American Heart Association guidelines (arm supported heart level, proper cuff size, etc). The site used an in-clinic BP measurement device provided by the Sponsor. Results were recorded in the subject's source documents and the eCRF.

Serum samples for the analysis of sUA were collected as part of the standard chemistry panel at the time points stipulated in Schedule of Study Procedures (Table 1). All samples were collected in accordance with standard laboratory procedures. Serum urate concentrations were determined using the enzymatic method as performed by the Central Laboratory and were blinded to the Sponsor and investigative site beginning at Day 1 through the Month 12/ET visits.

All subjects were instructed to take colchicine 0.6 mg QOD for prophylaxis. Colchicine began at the Screening Day −21 visit for all subjects. Subjects received colchicine 0.6 mg QOD through the Month 6 visit.

Colchicine was dispensed at the Screening visit (Day −21) and at the Month 3 visit. Alternatively, if colchicine was not tolerated by the subject, prednisone was provided at the investigator's discretion in accordance with the stated study guidelines.

At the Day −21 visit, in addition to dispensing the gout flare prophylaxis medication, the investigator instructed the subject to call the site in the event of a gout flare. If a gout flare occurred, the investigator provided additional gout flare treatment. Treatment choice were at the discretion of the investigator and in accordance with their practice guidelines but should excluded prescription and non-prescription NSAIDs or COX-2 inhibitors.

Subjects experiencing a gout flare could have the dose increased to colchicine 0.6 mg/day for the duration of the flare.

Gout flares were treated at the discretion of the investigator, as long as this treatment was in compliance with the prohibited medication guidance for this study. Subjects were instructed to contact the Investigator when they began to have a gout flare. The study site completed the Gout Flare Assessment Worksheet. An unscheduled visit was conducted if deemed appropriate by the Investigator. All subjects that experienced flares while on the study had the option to receive acute gout flare treatment if deemed appropriate by the Principal Investigator. The Investigator could also consult with the Medical Monitor for further discussion.

Subjects were assessed for gout flares from the time the ICF was signed and throughout the duration of the study. Subjects were instructed to call the Investigator as soon as they thought they were having a gout flare. The Gout Flare Assessment Worksheet was completed by the site personnel (ie, study coordinator, study nurse, or the investigator).

All gout flares were followed until complete resolution. Subjects were instructed to contact the site when flare had resolved, typically 7 to 10 days after onset. The study site contacted the subject 7 days after the initial report if the subject failed to report the end date of the gout flare. The attempts to contact the subject to obtain the end date of the gout flare were documented (2 documented telephone contact attempts).

Subjects were instructed to report the following information: the onset and end date of the flare, the kind of prophylaxis medication they were taking at the time of the event, whether or not the attack required medication (including type) and dates of treatment, the location of the flare, signs and symptoms regarding the flare including the severity, subjects' pain intensity (pain in rest), and the assessment of current gout flare compared to all previous gout flares in any joints. In addition, the subjects were asked an exploratory question in nature: the subject was asked during their current gout attack to indicate the degree of which the subject's physical mobility had been limited (on a scale of 0-10). The investigator reviewed the information provided and assessed whether or not he believed the subject experienced a gout flare and/or documented an alternative etiology.

The SAS System with the HP-Unix operating system was used to perform the statistical analyses. Unless otherwise specified, all statistical tests and CIs were two-sided and conducted at the 0.05 significance level. All computations were performed prior to rounding. Statistical significance was determined using p-values that were rounded to 3 decimal places. Unless otherwise specified, descriptive statistics on continuous variables consisted of the number of subjects (N), mean, standard deviation, minimum, 25th percentile, median, 75th percentile, and maximum.

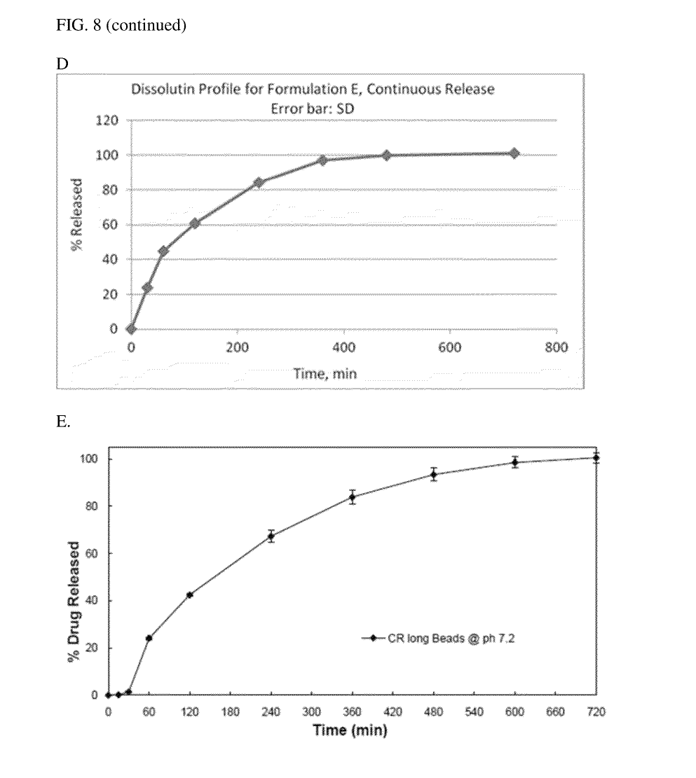

For the purpose of this example, study drug refers exclusively to the double-blind treatment namely: febuxostat 40 mg/80 mg QD or febuxostat 30 mg BID or placebo. Unless specified otherwise, all statistical tables by febuxostat 40 or 80 mg are presented combined with no summaries by separate doses. Febuxostat extended release capsules of the study, 40 mg and 80 mg, contained two types of beads: 20% of the total drug in immediate release (IR) beads and 80% of the total drug in delayed release beads designed to dissolve around pH ≧6.8 (“DR6.8”), i.e., the febuxostat 40 mg and 80 mg dosage forms included IR and DR Beads in a 2:8 ratio. The beads are filled into empty hard gelatin capsules and the capsule product shows a two-pulsatile dissolution profile. The composition of the IR granules in 80 mg capsules was 315 mg febuxostat/g bead, and the composition of the IR granules in the 40 mg capsules was 105 mg febuxostat/g bead (See FIG. 7).

Study Day 1 was defined as the date of the first dose of double-blind study drug, as recorded on the CRF dosing page. Subjects were dispensed double-blind study drug on the date of randomization and took the first dose on the same day. Other study days were defined relative to the Study Day 1.

Unless otherwise specified, the baseline value for a variable was defined as the last observation prior to receiving the first dose of study drug on Day 1.

A windowing convention was used to determine the analysis value for a given study visit and was applicable for all by-visit summaries and analyses, unless otherwise specified. The convention used for the analysis of efficacy and safety is summarized in Table 2.

TABLE 2
Visit Windows for Efficacy and Safety Variables

| | | | Efficacy and other safety | |
|---|---|---|---|---|
| Visit ID | Target Day | ECG | variables (study days) | SF-36 v2 |
| Baseline | ≦1 | ≦1 | ≦1 | ≦1 |
| Day 14 | 14 | NA | 2-22 | NA |
| Month 1 | 30 | NA | 23-59 | NA |
| Month 3 | 91 | NA | 60-137 | NA |
| Month 6 | 182 | NA | 138-228 | 2-273 |
| Month 9 | 273 | NA | 229-319 | NA |
| Month 12 | 364 | >1 | ≧320 | ≧274 |
| Final visit | Last day on study | >1 | >1 | >1 |

If a subject had more than 1 measurement in the same visit window, the measurement closest to the target day was used. If 2 measurements in the same window were of equal distance to the target day, the measurement that occurred after the target day was used. If 2 or more measurements occur on the same day, the last repeat value was used.

Demographic and baseline variables were summarized to assess the comparability of the treatment groups by randomization. Summary statistics were generated overall and by treatment group based on the FAS as well as the safety analysis set. No inferential statistics are presented.

Subjects' baseline renal function was categorized as severely impaired or moderately impaired based on their baseline eGFR. Subjects with eGFR ≧15 and <30 mL/min were classified as having severely impaired renal function, and those with eGFR ≧30 and ≦50 mL/min as having moderately impaired renal function. If the baseline eCLcr was missing, the average of the 3 screening eCLcr was used to determine the baseline renal function category.

All efficacy analysis was performed using the FAS population. The FAS consisted of all subjects who were randomized and received at least one dose of double-blind study medication

Missing data in all efficacy analyses was imputed with the last available postbaseline observation carrying forward (LOCF). A subject was included in the analysis of a specific efficacy variable only when there was both a baseline value and at least 1 value during the double-blind treatment period.

The primary efficacy variable was the change from Baseline to Month 12 in sCr. Summary statistics were presented for Baseline, postbaseline, and change from Baseline by treatment group at each visit. The primary analysis was based on the analysis of covariance (ANCOVA) model for change from baseline to Month 12 for the primary efficacy variable. The model included treatment as a factor, and the baseline sCr and prior use of an ARB or an ACEi (subjects taking an ARB or an ACEi or not taking an ARB or an ACEi) as covariates. The primary comparison was febuxostat 40 mg/80 mg QD versus placebo. The least squares (LS) mean, p-value and 2-sided 95% CI of treatment difference are provided.

Pair-wise comparison of febuxostat 30 mg BID to placebo was made with no adjustment for multiplicity.

Subgroup analyses for the primary efficacy variable were summarized for the following: baseline sCr (<2.0, 2.0 to <2.5, ≧2.5), baseline renal function (eGFR ≧15 and <30, eGFR ≧30 and ≦50 mL/min), baseline sUA (<9.0, 9.0 to <10.0, ≧10.0 mg/dL), and prior use of an ARB or an ACEi (ARB, ACEi, none).

In addition, the percentage of subjects with increases from Baseline to Month 6 and Month 12 greater than or equal to 25% and 50% in sCr was summarized by treatment group, and the treatment groups were compared by a Cochran-Mantel_Haenszel (CMH) test with prior use of an ARB or ACEi as a stratification variable.

The secondary efficacy variables for this study included change from baseline to Month 12 in eGFR using the MDRD formula; and percentage of subjects with sUA <6.0 mg/dL at Month 12.

An ANCOVA analysis similar to the primary analysis was used for analysis of eGFR. Subgroup analyses were also summarized for eGFR as for the primary endpoint.

The percentage of subjects with sUA <6 mg/dL at Month 12 was summarized by treatment group, and the treatment groups were compared by a CMH test with prior use of an ARB or ACEi as a stratification variable.

In addition, change from baseline and percent change from Baseline in sUA were summarized by visit and by treatment.

Additional efficacy variables for this study included: change from Baseline to Month 12 in sUA and change from Baseline to Month 6 and Month 12 in clinic systolic and diastolic BP.

For the additional efficacy variables, an ANCOVA analysis similar to the primary analysis was used. Unless otherwise specified, all ANCOVA analyses included treatment as a factor, the baseline value (when applicable) and prior use of an ARB or ACEi as covariates.

For the BP data, 3 sitting BP measurements were collected at each visit and the mean of the 3 measurements was used in all summary tables.

For all efficacy variables listed above, summary statistics are presented for Baseline, postbaseline, and change from Baseline by treatment group at each visit.

Each primary, secondary and additional efficacy variables analyzed at Month 12 was analyzed at Month 6 in a similar manner.

Summary statistics for the number and percentage of subjects experiencing a gout flare were summarized by treatment group within the following intervals: Day 1 to Month 2 (Day 1 to 60), Month 2 to 4 (Day 61 to 120), Month 4 to 6 (Day 121 to 180), Month 6 to 8 (Day 181 to 240), Month 8 to 10 (Day 241 to 300), Month 10 to 12 (>day 301, and overall Day 1 to Month 6, and Month 6 to Month 12. All gout flares with onset after Day 301 (including those reported <30 days since last dose) were included in the Month 10 to 12 interval.

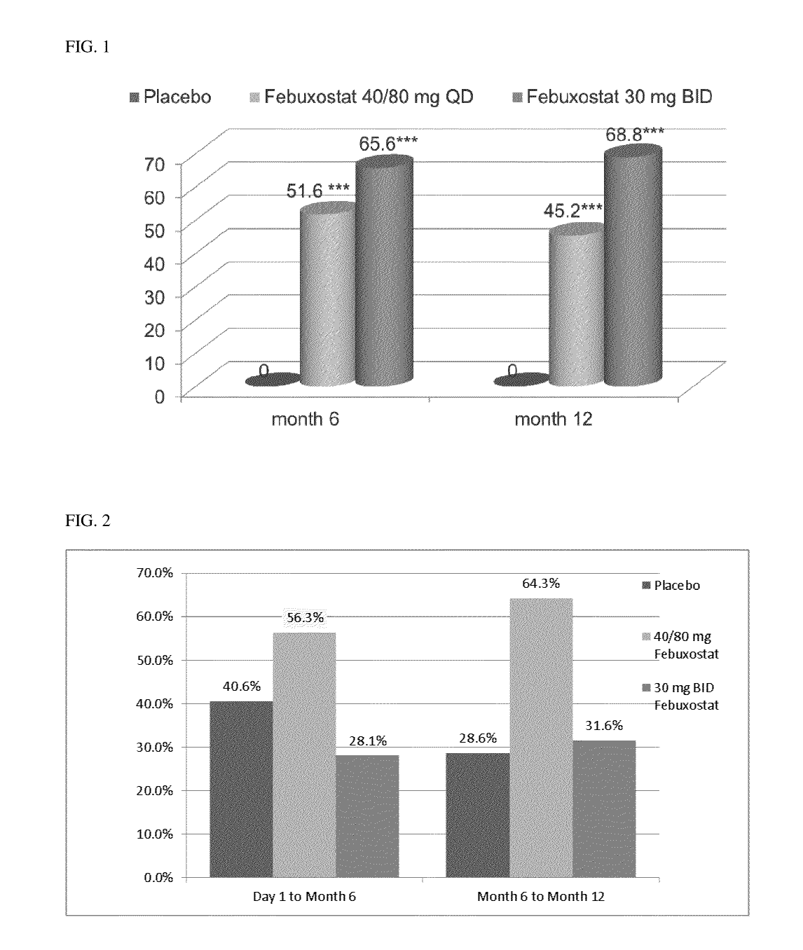

The percentage of subjects with sUA <6.0 mg/dL is summarized in Table 6 below. Statistical testing was only performed for Month 6 and Month 12. The percentage of subjects with sUA <6.0 mg/dL at both Month 6 and 12 were statistically significantly higher in both the febuxostat 30 mg BID and febuxostat 40/80 mg QD groups compared with placebo (p<0.001) (See FIG. 1 and Table 3).

TABLE 3
Percentage of Subjects with sUA <6.0
mg/dL to Month 12 in Full Analyses Set

| | Treatment Group | Febuxostat | Febuxostat |
|---|---|---|---|
| | Placebo | 30 mg BID | 40/80 mg QD |
| | (N = 32) | (N = 32) | (N = 32) |
| Visit | % (n/N) | % (n/N) | % (n/N) |
| Day 14 | 0 | 71.9 (23/32) | 33.3 (10/30) |
| Month 1 | 0 | 71.9 (23/32) | 35.5 (11/31) |
| Month 3 | 0 | 78.1 (25/32) | 58.1 (18/31) |
| Month 6 | 0 | 65.6 (21/32)* | 51.6 (16/31)* |
| Month 9 | 0 | 68.8 (22/32) | 58.1 (18/31) |
| Month 12 | 0 | 68.8 (22/32)* | 45.2 (14/31)* |

Source: Tables 15.2.3.1.
Note:
A subject was included in the analysis only when there were both baseline value and at least 1 postbaseline value. A missing value was imputed by the last available post-baseline value carrying forward.
*Indicates statistical significance vs placebo at p ≦ 0.05 level. Statistical testing was only performed for Month 6 and Month 12.

The analysis of the mean change from Baseline to Months 6 and 12 in sUA is presented in Table 4. At Month 12, the LS means for the change from Baseline in sUA were −0.15, −4.97, and −4.17 mg/dL for the placebo, febuxostat 30 mg BID, and febuxostat 40/80 mg QD groups, respectively. At both Month 6 and Month 12, statistically significant differences were seen between the placebo group and either febuxostat group. Further, a numerically greater reduction in sUA was achieved with febuxostat 30 mg BID compared with febuxostat 40/80 mg QD group.

TABLE 4
Analysis of Mean Change From Baseline to Month 12 Visit in Serum Urate

| | | Treatment Group | |
|---|---|---|---|
| | | Febuxostat 30 | Febuxostat |
| | Placebo | mg BID | 40/80 mg QD |
| Variable | (N = 32) | (N = 32) | (N = 32) |
| Serum urate change from | | | |
| Baseline at Month 6 (a) | | | |
| N | 32 | 32 | 31 |
| LS mean (SE) | 0.07 (0.304) | −5.08 (0.304) | −4.29 (0.309) |
| LS mean difference (SE) (b) | | −5.15 (0.428) | −4.36 (0.431) |
| p-value | | <0.001 | <0.001 |
| 2-Sided 95% CI | | −6.00, −4.30 | −5.21, −3.50 |
| Serum urate change from | | | |
| Baseline at Month 12 (a) | | | |
| N | 32 | 32 | 31 |
| LS mean (SE) | -0.15 (0.318) | −4.97 (0.318) | −4.17 (0.323) |
| LS mean difference (SE) (b) | | −4.82 (0.447) | −4.02 (0.451) |
| p-value | | <0.001 | <0.001 |
| 2-Sided 95% CI | | −5.71, −3.93 | (−4.91, −3.12) |

(a) Missing data was imputed as carrying forward the last postbaseline value,
(b) LS mean difference = LS mean difference from placebo group.
Note:
p-values were from an ANCOVA model with treatment as a factor, and the baseline value prior use of an ARB, ACEi, or none as covariates.

Overall, these data show that a numerically greater reduction in sUA was achieved with febuxostat 30 mg BID compared with febuxostat 40/80 mg QD group.

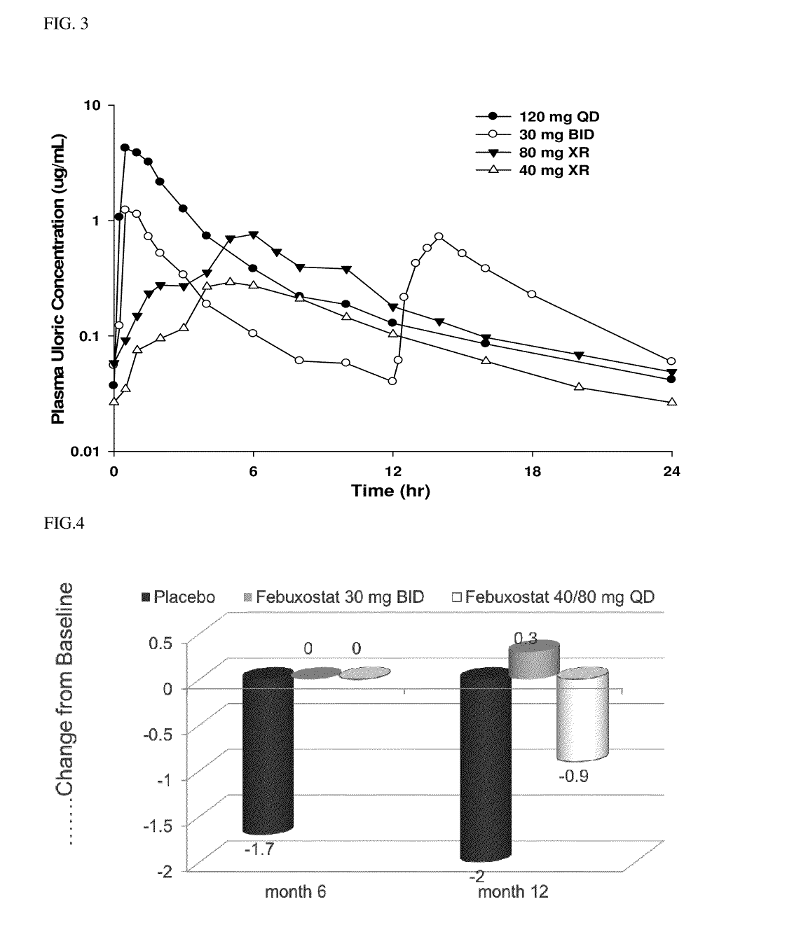

A summary of the percentage of subjects who experienced gout flares is presented in Table 5. In the subject group receiving 30 mg febuxostat IR BID, the percentage of subjects experiencing at least one gout flare was much lower than the percentage of subjects experiencing at least one gout flare in the 40/80 mg QD group and was smaller than or similar to the percentage of subjects experiencing at least one gout flare in the placebo group. During Months 1 to 6, 40.6%, 28.1% and 56.3% of subjects in the placebo, febuxostat 30 mg BID, and 40/80 mg QD groups experienced at least 1 gout flare. During Months 6 to 12, 28.6%, 31.6%, and 64.3% of subjects in the placebo, febuxostat 30 mg BID, and febuxostat 40/80 mg QD groups experienced at least 1 gout flare. The data for Months 1-6 and Months 6-12 are illustrated for each treatment group in FIG. 2.

Additionally, in the subject group receiving 40/80 mg febuxostat IR QD, the percentage of subjects experiencing at least one gout flare from months 6 to ≦8, the first two months after prophylactic treatment ended after month 6, increased by 279% relative to the percentage of subjects experiencing at least one gout flare from months 4 through 6 (50.0% vs. 17.9%). However, in the subject group receiving 30 mg febuxostat IR BID, the percentage of subjects experiencing at least one gout flare from months 6 to ≦8 increased by 84% relative to the percentage of subjects experiencing at least one gout flare from months 4 through 6 (26.3% vs. 14.3%). For the subject group receiving 30 mg febuxostat IR BID, the percentage of subjects experiencing at least one gout flare from months 6 to ≦8 was not significantly different from the percentage of subjects experiencing at least one gout flare from months 6 to ≦8 in the placebo group (26.3% vs. 19.0%).

TABLE 5
Percentage of Subjects With Gout Flares

| | Treatment Group | | |
|---|---|---|---|
| | Placebo | Febuxostat 30 | Febuxostat 40/80 |
| Interval | n/N (%) | mg BID n/N (%) | mg QD n/N (%) |
| Day 1 to Month 6 | 13/32 (40.6) | 9/32 (28.1)* | 18/32 (56.3) |
| ≦2 months | 9/32 (28.1) | 6/32 (18.8)* | 16/32 (50.0) |
| 2 to ≦4 months | 5/26 (19.2) | 3/28 (10.7) | 8/29 (27.6) |
| 4 to ≦6 months | 5/23 (21.7) | 3/21 (14.3) | 5/28 (17.9) |
| Month 6 to | 6/21 (28.6)* | 6/19 (31.6)* | 18/28 (64.3) |
| Month 12 | | | |
| 6 to ≦8 months | 4/21 (19.0)* | 5/19 (26.3) | 14/28 (50.0) |
| 8 to ≦10 months | 3/18 (16.7) | 1/17 (5.9) | 6/27 (22.2) |
| 10 to ≦12 months | 3/15 (20.0) | 2/17 (11.8) | 6/26 (23.1) |

Source: Tables 15.2.7.1
Note:
Percentage is relative to the number of subjects who have at least 1 day of drug exposure in the corresponding time interval.
* Indicates statistical significance vs febuxostat 40/80 mg QD at p ≦ 0.05 level.

The analysis of the primary efficacy variable, the change from Baseline to Month 12 in serum creatinine (sCr), is presented in Table 6. A summary of the mean sCr and mean change from Baseline to each visit is presented in Table 7.

At Month 12, the LS means for the change from Baseline in sCr were 0.19, 0.09, and 0.23 mg/dL for the placebo, febuxostat 30 mg BID, and febuxostat 40/80 mg QD groups, respectively. At both Month 6 and Month 12, there were no statistically significant differences between the placebo group and either febuxostat group (Table 6).

Although the differences were not statistically significant, there was a small favorable trend over time as compared to placebo in change in sCr for both the febuxostat 30 mg BID and febuxostat 40/80 mg QD groups. In the placebo group, mean sCr levels tended to increase while the levels were generally stable in both of the febuxostat groups.

TABLE 6
Analysis of Mean Change From
Baseline to Month 12 Visit in Serum Creatinine

| | Treatment Group | | |
|---|---|---|---|
| | | Febuxostat | Febuxostat |
| | Placebo | 30 mg BID | 40/80 mg QD |
| Variable | (N = 32) | (N = 32) | (N = 32) |

Serum creatinine change from Baseline at Month 6 (a)

| | | | |
|---|---|---|---|
| N | 32 | 32 | 31 |
| LS mean (SE) | 0.15 (0.062) | 0.04 (0.061) | 0.07 (0.061) |
| LS mean difference (SE) (b) | | −0.11 (0.087) | −0.08 (0.087) |
| p-value | | 0.213 | 0.356 |
| 2-Sided 95% CI | | −0.28, 0.06 | −0.25, 0.09 |

Serum creatinine change from Baseline at Month 12 (a)

| | | | |
|---|---|---|---|
| N | 32 | 32 | 31 |
| LS mean (SE) | 0.19 (0.094) | 0.09 (0.093) | 0.23 (0.094) |
| LS mean difference (SE) (b) | | −0.10 (0.134) | 0.04 (0.133) |
| p-value | | 0.459 | 0.789 |
| 2-Sided 95% CI | | −0.37, 0.17 | −0.23, 0.30 |

Source: Table 15.2.1.2.1.
(a) Missing data was imputed as carrying forward the last postbaseline value.
(b) LS mean difference = LS mean difference from placebo group.

TABLE 7
Change from Baseline to Each Visit in Serum Creatinine - Full Analyses Set

| | Treatment Group | | | | | | | | |
|---|---|---|---|---|---|---|---|---|---|
| | Placebo (N = 32) | | | Febuxostat 30 mg BID (N = 32) | | | Febuxostat 40/80 mg QD (N = 32) | | |
| Variable | | Mean | Change | | Mean | Change | | Mean | Change |
| Visit | n | (SD) | (SD) | n | (SD) | (SD) | n | (SD) | (SD) |

Serum Creatinine (mg/dL)

| | | | | | | | | | |
|---|---|---|---|---|---|---|---|---|---|
| Baseline | 32 | 2.52 (0.915) | | 32 | 2.09 (0.451) | | 31 | 2.21 (0.675) | |
| Day 14 | 31 | 2.49 (0.828) | 0.01 (0.245) | 32 | 2.18 (0.548) | 0.09 (0.297) | 30 | 2.30 (0.811) | 0.08 (0.313) |
| Month 1 | 32 | 2.58 (0.929) | 0.06 (0.234) | 32 | 2.23 (0.588) | 0.13 (0.44) | 31 | 2.25 (0.749) | 0.05 (0.276) |
| Month 3 | 32 | 2.59 (1.002) | 0.07 (0.354) | 32 | 2.18 (0.569) | 0.09 (0.382) | 31 | 2.24 (0.806) | 0.04 (0.288) |
| Month 6 | 32 | 2.67 (1.014) | 0.15 (0.348) | 32 | 2.12 (0.527) | 0.03 (0.364) | 31 | 2.26 (0.814) | 0.06 (0.313) |
| Month 9 | 32 | 2.68 (1.066) | 0.16 (0.365) | 32 | 2.21 (0.640) | 0.11 (0.426) | 31 | 2.22 (0.940) | 0.01 (0.481) |
| Month 12 | 32 | 2.74 (1.134) | 0.23 (0.481) | 32 | 2.13 (0.676) | 0.04 (0.478) | 31 | 2.40 (1.147) | 0.19 (0.658) |

Source: Tables 15.2.1.1.1.
Note:
A subject was included in the analysis only when there were both Baseline value and at least 1 postbaseline value. A missing value was imputed by the last available postbaseline value carrying forward.

The change from Baseline to Month 12 in sCr was summarized by the following subgroups: Baseline sCr (<2.0, 2.0 to <2.5, ≧2.5), baseline renal function (eGFR ≧15 and <30 [severe impairment], eGFR ≧30 and ≦50 mL/min [moderate impairment]), baseline sUA (<9.0, 9.0 to <10.0, ≧10.0 mg/dL), and prior use of ARB or ACEi (ARB, ACEi, none).

At both Months 6 and 12, subjects with moderate renal impairment who received febuxostat 30 mg BID had generally minimal changes in mean sCr levels compared with small increases in subjects who received placebo or febuxostat 40/80 mg. This difference was not apparent among subjects with severe renal impairment.

Additionally, at both Months 6 and 12, subjects with prior and/or current use of ARB who had received febuxostat 30 mg BID or febuxostat 40/80 mg generally had minimal changes or a slight improvement in mean sCr levels compared with large increases in subjects who received placebo. In subjects with prior and/or current use of ACEi, slight improvements in sCr were seen at Months 6 and 12 in the febuxostat 30 mg BID group, with minimal changes seen in the febuxostat 40/80 mg QD and placebo groups. In subjects with no prior and/or current use ARB or ACEi, mean sCr increased in all treatment groups.

In subjects with sCr 2.0 to <2.5 mg/dL at Baseline, there was a small favorable trend over time compared with placebo in change in sCr for both the febuxostat 30 mg BID and febuxostat 40/80 mg QD groups. In subjects with sCr <2.0 or ≧2.5 mg/dL at Baseline, no differences were seen across treatment groups.

In subjects with sUA of 9.0 to <10.0 mg/dL at Baseline there was a small favorable trend over time as compared to placebo in change in sCr for both the febuxostat 30 mg BID and febuxostat 40/80 mg QD groups. In subjects with sUA <9.0 or ≧10.0 mg/dL at Baseline, no differences were seen across treatment groups.

Overall, these data show that a favorable trend in renal function preservation was observed with both febuxostat 30 mg BID and febuxostat 40/80 mg QD groups compared with placebo, although the differences were not statistically significant. Moreover, the febuxostat 30 mg BID showed better renal function preservation than febuxostat 40/80 mg QD.

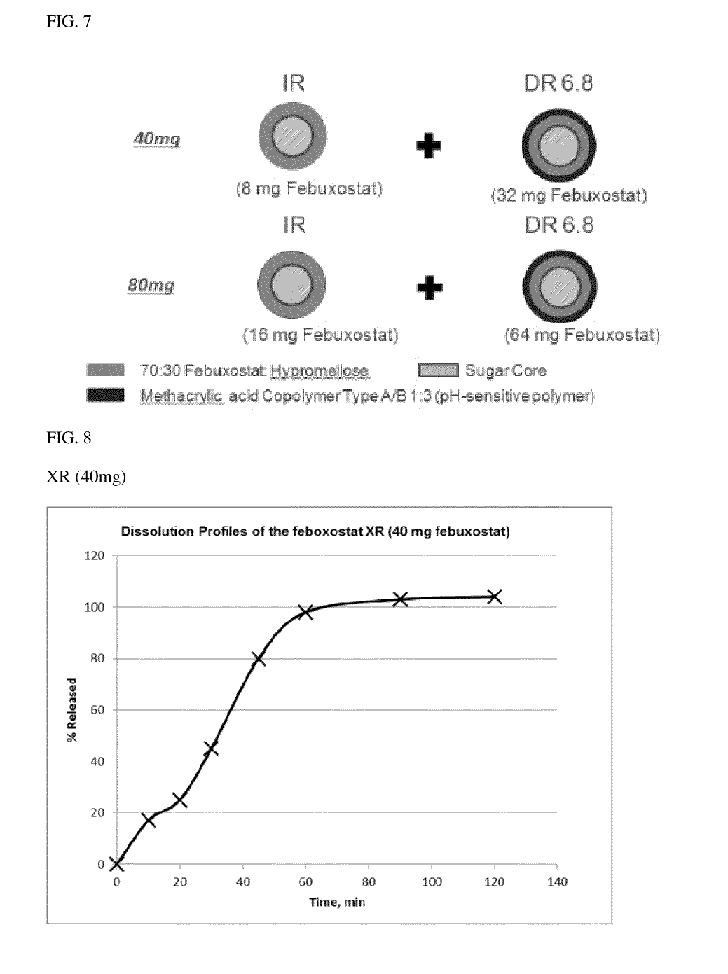

The analysis of the change from Baseline to Month 12 in eGFR using the MDRD formula is presented in Table 8 and illustrated in FIG. 4. A summary of the mean eGFR and mean change from Baseline to each visit in eGFR using the MDRD formula is presented in Table 9.

At Month 12, the LS mean for the change from Baseline in eGFR using the MDRD formula was −2.05, 0.33, and −0.086 mL/min/1.73 m² for the placebo, febuxostat 30 mg BID, and febuxostat 40/80 mg QD groups, respectively.

Although the differences were not statistically significant, there was a small favorable trend over time as compared to placebo in eGFR for both the febuxostat 30 mg BID and febuxostat 40/80 mg QD groups. Moreover, the febuxostat 30 mg BID showed better renal function preservation than febuxostat 40/80 mg QD.

TABLE 8
Analysis of Change From Baseline to Month 12 in
eGFR (mL/min/1.73 m²) Using the MDRD Formula

| | Treatment Group | | |
|---|---|---|---|
| | | Febuxostat | Febuxostat |
| Visit | Placebo | 30 mg BID | 40/80 mg QD |
| Evaluation | (N = 32) | (N = 32) | (N = 32) |

eGFR (mL/min/1.73 m²)
Baseline

| | | | |
|---|---|---|---|
| N | 32 | 32 | 31 |
| LS mean (SE) | 29.31 (1.461) | 34.14 (1.461) | 34.08 (1.484) |
| LS mean difference | | 4.84 (2.052) | 4.77 (2.068) |
| (SE) (a) | | | |
| p-value | | 0.021 | 0.023 |
| 2-sided 95% CI | | 0.76, 8.91 | 0.66, 8.88 |

Change from Baseline at Month 6 (b)

| | | | |
|---|---|---|---|
| N | 32 | 32 | 31 |
| LS mean (SE) | −1.71 (0.976) | 0.03 (0.955) | 0.04 (0.969) |
| LS mean difference | | 1.74 (1.374) | 1.75 (1.383) |
| (SE) (a) | | | |
| p-value | | 0.208 | 0.209 |
| 2-sided 95% CI | | −0.99, 4.47 | −1.00, 4.50 |

Change from Baseline at Month 12 (b)

| | | | |
|---|---|---|---|
| N | 32 | 32 | 31 |
| LS mean (SE) | −2.05 (1.198) | 0.33 (1.172) | −0.86 (0.190) |
| LS mean difference | | 2.38 (1.687) | 1.19 (1.698) |
| (SE) (a) | | | |
| p-value | | 0.162 | 0.485 |
| 2-sided 95% CI | | −0.97, 5.73 | −2.18, 4.57 |

Source: Table 1.5.2.2.2.
(b) LS mean difference = LS mean difference from placebo group.
(a) Missing data was imputed as carrying forward the last post-baseline value.
Note:
p-values were from an ANCOVA model with treatment as a factor, and the baseline value and prior use of an ARB, ACEi, or none as covariates. Prior use of ARB/ACEi/none is based on IVRS data.

TABLE 9
Change (SD) from Baseline to Month 12 in eGFR (mL/min/1.73 m²) Using the MDRD Formula - Full Analyses Set

| | Treatment Group | | | | | | | | |
|---|---|---|---|---|---|---|---|---|---|
| | Placebo (N = 32) | | | Febuxostat 30 mg BID (N = 32) | | | Febuxostat 40/80 mg QD (N = 32) | | |
| Variable | | Mean | Change | | Mean | Change | | Mean | Change |
| Visit | n | SD) | (SD) | n | (SD) | (SD) | n | (SD) | (SD) |

eGFR using MDRD formula (mL/min/1.73 m²)

| | | | | | | | | | |
|---|---|---|---|---|---|---|---|---|---|
| Baseline | 32 | 29.40 (7.957) | | 32 | 34.24 (7.145) | | 31 | 34.21 (9.284) | |
| Day 14 | 31 | 29.28 (7.520) | −0.33 (3.285) | 32 | 33.25 (8.042) | −0.99 (4.958) | 29 | 33.87 (10.259) | −0.52 (4.646) |
| Month 1 | 32 | 28.86 (8.067) | −0.55 (2.937) | 31 | 32.99 (8.305) | −1.48 (5.938) | 31 | 33.91 (10.482) | −0.29 (5.083) |
| Month 3 | 32 | 28.91 (8.546) | −0.49 (4.927) | 32 | 33.52 (9.005) | −0.72 (6.654) | 31 | 34.34 (10.474) | 0.14 (4.968) |
| Month 6 | 32 | 27.98 (8.379) | −1.42 (4.138) | 32 | 34.18 (8.004) | −0.06 (6.345) | 31 | 34.18 (11.067) | −0.03 (5.204) |
| Month 9 | 32 | 28.09 (9.040) | −1.31 (4.548) | 32 | 33.33 (9.468) | −0.91 (6.884) | 31 | 35.39 (10.930) | 1.18 (6.783) |
| Month 12 | 32 | 27.66 (8.890) | −1.74 (4.994) | 32 | 34.56 (9.382) | 0.32 (6.789) | 31 | 33.38 (11.949) | −0.82 (7.619) |

Source: Table 15.2.2.1.
Note:
A subject was included in the analysis only when there were both baseline value and at least 1 postbaseline value. A missing value was imputed by the last available postbaseline value carrying forward..

The change from Baseline to Month 12 in eGFR using the MDRD was summarized by the following subgroups: baseline serum creatinine (<2.0, 2.0 to <2.5, ≧2.5), baseline renal function (eGFR ≧15 and <30 [severe impairment], eGFR ≧30 and ≦50 mL/min [moderate impairment]), Baseline sUA (<9.0, 9.0 to <10.0, ≧10.0 mg/dL), and prior use of ARB or ACEi (ARB, ACEi, none).

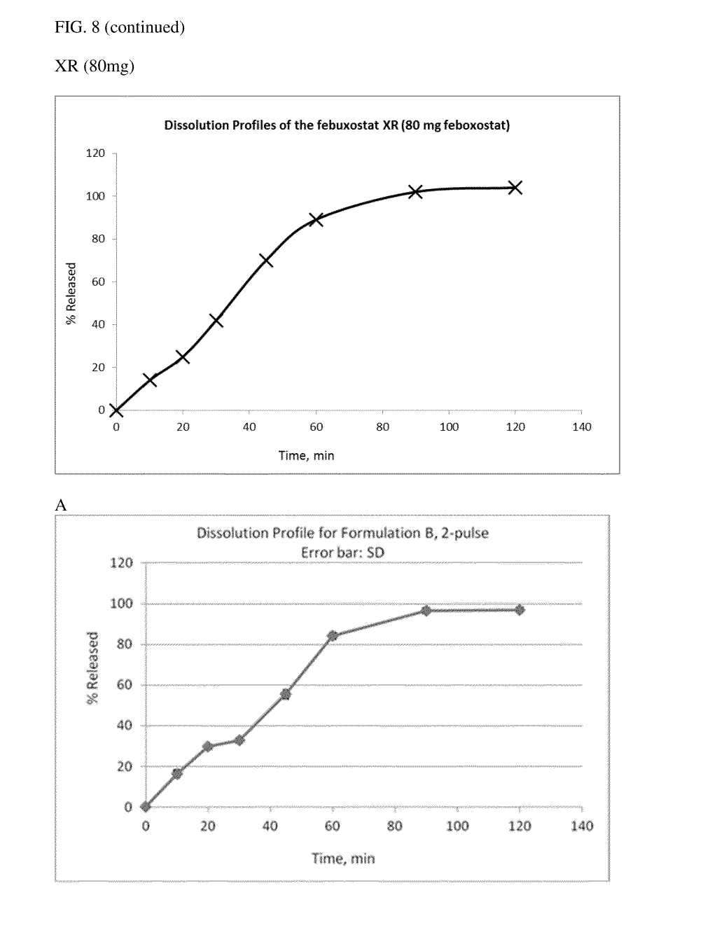

FIG. 5 illustrates the mean change from Baseline eGFR by MDRD at Months 6 and 12 by baseline renal function. At Month 12, subjects with moderate renal impairment who received febuxostat 30 mg BID had had a small improvement in mean eGFR levels compared with small decreases in mean eGFR levels in subjects who received placebo or febuxostat 40/80 mg. This difference between treatment groups was not apparent among subjects with severe renal impairment.

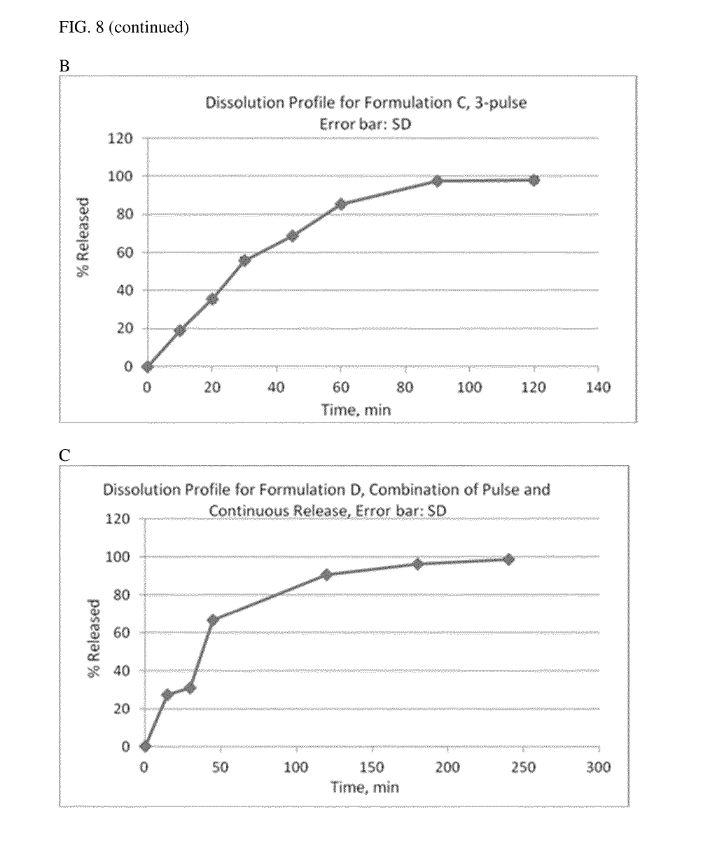

FIG. 6 illustrates the mean change from Baseline in eGFR by MDRD at Months 6 and 12 by prior use of ARB, ACEi, or none.

In the subgroup of subjects with prior use of ARB, those who had received febuxostat 30 mg BID generally had modest improvements in eGFR levels over time compared with the moderate to large decreases in eGFR in subjects who received placebo or febuxostat 40/80 mg. In the subgroup of subjects with prior use of ACEi, slight improvements in eGRF over time were observed in the febuxostat 30 mg BID and 40/80 mg QD groups, with minimal changes placebo group. In the subgroup of subjects with no prior use ARB or ACEi, changes in mean eGFR were generally similar for all treatment groups.

In subjects with serum creatinine 2.0 to <2.5 mg/dL at Baseline, there was a small favorable trend over time compared with placebo in change in eGFR for both the febuxostat 30 mg BID and febuxostat 40/80 mg QD groups. In subjects with serum creatinine <2.0 or ≧2.5 mg/dL at Baseline, no differences were seen across treatment groups.

In subjects with sUA of 9.0 to <10.0 mg/dL at Baseline, there was a small favorable trend over time compared with placebo in change in eGFR for both the febuxostat 30 mg BID and febuxostat 40/80 mg QD groups. In subjects with sUA ≧10.0 mg/dL at Baseline, this small trend was apparent only in the febuxostat 30 mg BID group. In subjects with sUA <9.0, no differences were seen across treatment groups.

### Example 2

### Efficacy Comparison of 30 Mg Febuxostat IR BID Dosing with 40 and 80 Mg Febuxostat IR QD Dosing

In a clinical study (Study TMX-99-001) conducted in healthy volunteers, the percentage decrease in serum urate levels following oral administration of 30 mg febuxostat immediate release (IR) doses BID for 14 days was similar to that observed following administration of a single oral 120 mg febuxostat IR dose QD for 14 days (Table 10). The time for which the plasma concentration of febuxostat was ≧0.1 μg/mL was also determined (Table 10)

TABLE 10
Duration of Febuxostat Plasma concentration
≧ 0.1 μg/mL and Percentage reduction in serum
urate on Day 14 following different doses of Febuxostat.

| | Time (hr) Plasma | Reduction |
|---|---|---|
| | Conc. > 0.1 | (%) in Serum |
| Dose (mg) | μg/mL on Day 14 | Urate from Baseline |
| 30 mg IR QD | 5 | 32.7 |
| 120 mg IR QD | 15 | 63.4 |
| 30 mg IR BID | 17 | 62.1 |
| 80 mg XR QD | 16 | 58.9 |
| 40 mg XR QD | 10 | 42.4 |
| (simulated) | | |

As shown in the Table 10, in the TMX-99-001 study, we found that the extent of serum urate reduction with 30 mg BID was similar to 120 QD and was also correlated to the time (approx. 15-16 hrs) at or above 0.1 μg/mL. Therefore, when simulation and modeling was done for designing 80 mg XR based on 30 mg BID, the time (hrs) at or above 0.1 μg/mL was used as a design factor.

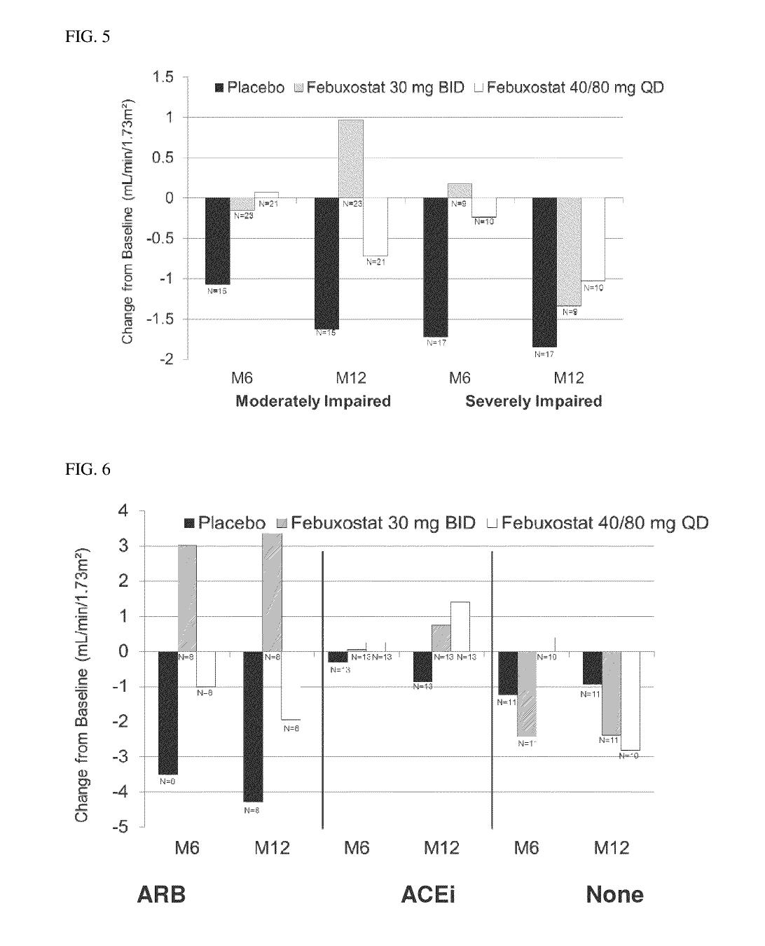

Similarly, in another clinical study conducted in healthy volunteers (Study TMX-67-106), the percentage decrease in serum urate levels following oral administration of a single oral 80 mg febuxostat XR (“febuxostat XR” means specific formulation as is described in Example 5) QD for 14 days was similar to that observed after oral administration of 30 mg febuxostat IR doses BID for 14 days (Table 10). FIG. 3 shows the febuxostat plasma concentration profiles of 30 mg BID, 80 mg XR, 120 mg QD and 40 mg XR (simulated). For all three dosing regimens (30 mg febuxostat IR doses BID, 120 mg febuxostat IR dose QD, and 80 mg febuxostat XR QD), plasma concentration of febuxostat was observed to remain at or above 0.1 μg/mL for a similar extended period of time, about 16 hours (FIG. 3 and Table 10).

Since administration of 30 mg febuxostat IR BID for 14 days resulted in a similar reduction in sUA and a similar time period at which plasma febuxostat concentration was ≧0.1 μg/mL as did administration of 80 mg febuxostat XR QD for 14 days, the 30 mg febuxostat IR BID dosing and the 80 mg febuxostat XR QD dosing are equivalent with respect to efficacy.

Similarly, simulations of the febuxostat plasma concentration time course for administration of a 40 mg febuxostat XR dosage form QD for 14 days resulted in a predicted percentage decrease in serum urate levels following oral administration of a single oral 80 mg febuxostat XR QD for 14 days and a predicted time at which plasma concentration of febuxostat would remain at or above 0.1 μg/mL similar to values observed after oral administration of 30 mg febuxostat IR doses BID for 14 days (Table 10 and FIG. 3). These simulations suggest that the 30 mg febuxostat IR BID dosing and the 40 mg febuxostat XR QD dosing are also similar with respect to efficacy.

The plasma concentration profile of febuxostat, as shown in FIG. 3, in conjunction with the data regarding the gout flare reduction effect, as shown in FIG. 2, has indicated that dosing regimens such as the 30 mg febuxostat IR BID and the 80 mg febuxostat XR QD which produce fluctuations in the subject's febuxostat plasma concentration profile within a certain value for a period after administration up to 24 hours, result in a significant reduction of the number/percentage of subjects with gout flares. Such fluctuations were quantified as the ratio of the maximum plasma febuxostat concentration (Cₘₐₓ) to the minimum plasma febuxostat concentration profile (Cₘᵢₙ) for a period of from administration to 24 hours. The Cmax/Cmin of the 30 mg febuxostat IR BID, the 80 mg febuxostat XR QD having gout flare reduction effect are 49.7 and 24.4, respectively while 40 mg febuxostat IR QD (data not shown) having no gout flare reduction effect is 88.3. These results show that the formulations, after administration to a subject in need of treatment of a xanthine oxidoreductase inhibitor, which produce in the subject Cₘₐₓ/Cₘᵢₙ of febuxostat less than or equal to 80, 70, 60 or 50, in particular ≦50, for a period from administration to 24 hours, result in a significant reduction of the number/percentage of subjects with gout flares.

In vitro dissolution profiles of the 80 mg febuxostat XR show that modified release dosage forms having in vitro febuxostat dissolution profile of 20-60% released after 30 min and 70-100% released after 60 min of the total amount of febuxostat in the dosage form measured using USP Apparatus I in 900 mL of 50 mM phosphate buffer at pH 6.90 with stirring at 100 rpm at 37° C. result in a significant reduction of the number/percentage of subjects with gout flares.

### Example 3

### Comparison of Modified Release and Immediate Release Febuxostat Dosage Forms

A randomized, double blind, multicenter, active-controlled study to evaluate the efficacy and safety of febuxostat 80 mg XR, 40 mg XR, 80 mg IR and 40 mg IR in subjects with gout is designed and performed.

A total of 200 eligible subjects are enrolled and randomized into 1 of 4 treatment groups. The overall duration of the study is 3 month.

The study consists of a Day −21 Screening Visit, a 3-week Washout Period (Day −21 to Day −1) for subjects currently receiving urate-lowering therapy (ULT), a Day −4 Screening Visit, Day 1 Randomization Visit, and a 3-month Double-Blind Treatment Period.

All subjects must have a serum urate level (sUA) >7.0 mg/dL at Day −4, and an estimated glomerular filtration rate (eGFR) ≧30 mL/min and <60 mL/min at Screening Day −21 for subjects on ULT and at screening Day −4 for subjects not on ULT.

At the Day −21 Screening Visit, subjects currently on ULT discontinue the ULT treatment and begin receiving 0.6 mg colchicine every other day (QOD) for gout flare prophylaxis. Subjects not previously taking ULTs prior to the study complete screening procedures at the Day −4 Visit and begin colchicine 0.6 mg QOD on the Randomization Visit (Day 1). Alternatively, if colchicine is not tolerated, naproxen 250 mg BID may be administered with lansoprazole 15 mg QD or with an appropriate dose of another proton pump inhibitor (PPI).

At the Day −4 Screening Visit, subjects have their blood drawn to determine their baseline sUA level for qualification into the randomized Double-Blind Treatment Period. If the subject's sUA level is >7.0 mg/dL and all other admission criteria are met, the subject returns for the Day 1 Randomization Visit.

At the day 1 Randomization visit, subjects are randomly assigned to 1 of 4 treatment groups in a 1:1:1:1 ratio to receive daily febuxostat 40 mg XR, 80 mg XR, 40 mg IR, or 80 mg IR for the study duration of 3 months. Randomization is stratified at baseline using 2 strata: subjects taking a urate-lowing therapy (ULT) at the time of screening visit; or subjects not taking a ULT. Estimated GFR (eGFR) calculation based on MDRD is performed at all visits. Moderate renal impairment is defined as an eGFR from 30 mL/min to 59 mL/min, inclusive.

Site personnel will use an interactive voice-activated response system (IVRS) to receive subject numbers and assign subjects to 1 of 4 treatment groups based on the randomization schedule provided by Takeda.

All subjects receive 0.6 mg colchicine QOD for gout flare prophylaxis for the study duration of 3 months. Alternatively, if colchicine is not tolerated, naproxen 250 mg BID is administered with lansoprazole 15 mg QD or with an appropriate dose of another PPI. In instances when subjects should not receive colchicine or naproxen, other NSAIDs or prednisone are provided in accordance with the Prohibited Concomitant Medications guidelines for the study.

Following the Day 1 Randomization Visit, four additional visits are required at Week 2, Month 1, 2, and 3 (final visit). The sponsor, the investigator, study coordinator, and subject are blinded to sUA levels after the Day 4 Visit through the end of the study.

All subjects are instructed to report the occurrence of pregnancy and any AEs or serious adverse events (SAEs) that occurred during the study and within 30 days following the discontinuation of study drug.

Starting from Day 1, subjects orally self-administer one capsule of their assigned febuxostat dosage form for the duration of the 3 month study.

Subjects experiencing a gout flare may have the dose increased to colchicine 0.6 mg/day for the duration of the flare. Gout flares may also be treated at the discretion of the investigator, as long as this treatment is in compliance with the prohibited medication for this study. Subjects are instructed to contact the Investigator when they begin to have a gout flare. The study site completes the Gout Flare Assessment Worksheet. An unscheduled visit is conducted when deemed appropriate by the Investigator. All subjects that experience flares while on the study have the option to receive acute gout flare treatment when deemed appropriate by the Principal Investigator.

The primary endpoint of the study is the proportion of subjects whose serum urate level is <6.0 mg/dL at Month 3 visit. The primary comparison is febuxostat 40 mg XR QD and 80 mg XR QD versus febuxostat 40 mg IR QD and 80 mg IR QD, respectively. Pairwise comparisons between treatment groups are made using Fisher's exact test

A secondary endpoint of the study is the percent reduction from baseline to Month 3 in serum urate. The percent reduction from baseline to Month 3 in serum urate levels is tested using an analysis of variance (ANOVA) model with a factor for treatment and baseline sUA and prior use of ULT (subjects taking a ULT prior to screening or not taking a ULT prior to screening) as covariates. Summary statistics will be presented for Baseline, Month 3, and change from Baseline to Month 3 by treatment group.

Another endpoint of the study is the percentage of subjects with gout flares during the study period.

Fewer subjects receiving febuxostat 40 mg XR experience gout flares during the study period than subjects receiving febuxostat 40 mg IR. Fewer subjects receiving febuxostat 80 mg XR experience fewer gout flares during the study period than subjects receiving febuxostat 80 mg IR.

The gout flare rate of the 40 mg febuxostat XR group is about 5% to about 50% less than the gout flare rate of the 40 mg febuxostat IR group, about 10% to about 30% less than the gout flare rate of the 40 mg febuxostat IR group. The gout flare rate of the 80 mg febuxostat XR group is about 5% to about 50% less than the gout flare rate of the 80 mg febuxostat IR group, about 10% to about 30% less than the gout flare rate of the 80 mg febuxostat IR group.

The gout flare rate of the 40 mg febuxostat XR group for the 3 month study period is about 5% to about 50%, about 10% to about 40%, about 15% to about 35%.

The gout flare rate of the 40 mg febuxostat IR group for the 3 month study period is about 5% to about 65%, about 10% to about 50%, about 15% to about 50%.

The gout flare rate of the 80 mg febuxostat XR group for the 3 month study period is about 5% to about 50%, about 10% to about 40%, about 15% to about 35%.

The gout flare rate of the 80 mg febuxostat IR group for the 3 month study period is about 5% to about 65%, about 10% to about 50%, about 15% to about 50%.

### Example 4

### Comparison of Modified Release without Dose-Escalation and Immediate Release with Dose-Escalation Febuxostat Dosage Forms

A randomized, double blind, multicenter, active-controlled study to evaluate the efficacy and safety of febuxostat 20 mg, 25 mg, 30 mg, 35 mg or 40 mg XR once daily and 10-20-40 mg IR once daily with dose-escalation in subjects with gout or hyperuricemia is designed and performed.

A total of 200 or the like eligible subjects are enrolled and randomized into 1 of 2 treatment groups. The overall duration of the study is 22 weeks or the like.

The endpoint of the study is the proportion of subjects whose serum urate level is <6.0 mg/dL. The comparison is febuxostat 20 mg, 25 mg, 30 mg, 35 mg or 40 mg XR once daily without dose-escalation versus febuxostat 10-20-40 mg IR once daily with dose-escalation.

Safety of the febuxostat 20 mg, 25 mg, 30 mg, 35 mg or 40 mg XR once daily group is within the permissible range and the proportion of subjects whose serum urate level is <6.0 mg/dL in the febuxostat 20 mg, 25 mg, 30 mg, 35 mg or 40 mg XR once daily group without dose-escalation is almost the same or higher than that in the febuxostat 10-20-40 mg IR once daily group with dose-escalation.

### Example 5

### Formulation Systems

Representative types of modified release dosage form are the membrane controlled system such as the febuxostat XR formulation used in the Examples 2 and 3, and the matrix system or the osmotic pump system as described before.

Another four membrane controlled system formulations (Formulations B, C, D, and E) of the same type as the febuxostat XR formulation, four matrix system formulations (Formulations 1-4) of matrix system were prepared and analyzed.

The febuxostat XR formulation and Formulation B are membrane controlled systems comprising a combination of an immediate release form and a delayed release form using a pH 6.8 enteric coating as the fill in capsules. The XR and B formulations are each characterized by a two pulsatile dissolution profile.

The 40 mg and 80 mg febuxostat XR capsules contain two types of beads: 20% drug in immediate release (IR) beads and 80% drug in delayed release beads designed to dissolve at pH ≧6.8 (DR6.8). Two strengths of IR beads were prepared, 105 mg febuxostat/g total bead for the 40 mg capsules and 315 mg febuxostat/g total bead for the 80 mg capsules. Compositions of the three types of beads and the 40 mg and 80 mg XR capsules are shown in Tables 11A and 11B below. Formulation B capsules differ from the XR capsules by the ratio of IR:DR6.8 beads in the hard gelatin capsules, 30% IR beads:70% DR6.8 beads, but are otherwise identical.

The IR beads are prepared by layering febuxostat onto sugar spheres using hypromellose as a binder. Enteric polymeric coating (1:3 ratio of methacrylic acid copolymer type AIB) is applied to the 315 mg/g beads to obtain DR6.8 beads with a theoretical potency of 277 mg febuxostat/g total bead. FIG. 7 illustrates the febuxostat IR and DR6.8 beads. Separately, the IR beads and the DR6.8 beads are lubricated with talc. Empty hard gelatin capsules are filled with the appropriate blend of lubricated beads with a dual-header filler, and show a two-pulsatile dissolution profile.

TABLE 11A
Composition of IR and DR6.8 beads for
40 mg and 80 mg febuxostat XR capsules and Formulation B
Composition of IR Beads (Theoretical potency 315 mg/g)^{a}

| | Compendial | | Unit Composition |
|---|---|---|---|
| Component Name | Reference | Function | (mg/g) |
| Febuxostat | In-house | Active | 315.0 |
| | (NDA 21-856) | | |
| Hypromellose | USP | Binder | 135.0 |
| Sugar Spheres 30-35 mesh | NF | Core | 522.5 |
| Hypromellose | USP | Seal coat layer | 27.5 |
| Purified Water (b) | NF | Solvent | — |

(a) Constitute IR portion of 80 mg capsules and are used to manufacture DR6.8 beads used in both 40 and 80 mg capsules.
(b) Removed during processing.
Composition of IR Beads (Theoretical potency 105 mg/g)^{a}

| | Compendial | | Unit Composition |
|---|---|---|---|
| Component Name | Reference | Function | (mg/g) |
| Febuxostat | In-house | Active | 105.0 |
| | (NDA 21-856) | | |
| Hvpromellose | USP | Binder | 45.0 |
| Sugar Spheres 30-35 mesh | NF | Core | 807.5 |
| Hypromellose | USP | Seal coat layer | 42.5 |
| Purified Water (b) | NF | Solvent | — |

(a) Constitute IR portion of 40 mg capsules.
(b) Removed during processing.
Composition of DR6.8 Beads (Theoretical Potency 277 mg/g)^{a}

| | Compendial | | Unit Composition |
|---|---|---|---|
| Component Name | Reference | Function | (mg/g) |
| Febuxostat IR Beads (315 mg/g) | In-house | Active | 879.3 |
| Methacrylic add copolymer Type A | NF | Enteric polymer | 27.4 |
| Methacrylic acid copolymer Type B | NF | Enteric polymer | 82.3 |
| Triethyl citrate | NA | Plasticizer | 11.0 |
| Acetone (b) | NF | Solvent | — |
| Purified Water (b) | NF | Solvent | — |
| Isopropanol (b) | NF | Solvent | — |

(a) Constitute DR portion of both 40 mg and 80 mg capsules.
(b) Removed during processing.

TABLE 11B
Composition of 40 mg and 80 mg febuxostat XR capsules
Composition of Febusostat Capsules in terms of Filling Components

| | | 80 mg | 40 mg |
|---|---|---|---|
| | Compendial | Capsules | Capsules |
| Component Name | Reference | (mg/capsule) | (mg/capsule) |
| Febuxostat IR Beads | NA | 50.8 | — |
| (315 mg/g) | | | |
| (Composition in | | (16.0) | — |
| terms of febuxostat) | | | |
| Febuxostat IR Beads | NA | — | 76.2 |
| (105 mg/g) | | | |
| (Composition in | | — | (8.0) |
| terms of febuxostat) | | | |
| Talc | NF | 0.5 | 0.8 |
| DR6.8 Beads (277 mg/g) | NA | 231.0 | 115.5 |
| (Composition in | | (64.0) | (32.0) |
| terms of febuxostat) | | | |
| Talc | NF | 2.3 | 1.2 |
| Hard gelatin capsule, | In-house | 1 capsule | 1 capsule |
| gray, Size 1 | | | |

Composition of Febuxostat Capsules in terms of Underlying Ingredients

| | | 80 mg | 40 mg |
|---|---|---|---|
| | | Capsules | Capsules |
| Component Name | Type | (mg/capsule) | (mg/capsule) |
| Febuxostat | NA | 79.98 | 39.99 |
| Hypromellose | 2910 | 41.26 | 23.17 |
| Sugar Spheres | Suglets PF 006 | 132.7 | 114.6 |
| Methacrylic acid | Endragit L100 | 6.329 | 3.165 |
| copolymer Type A | | | |
| Methacrylic acid | Endragit S100 | 19.01 | 9.506 |
| copolymer Type B | | | |
| Triethyl citrate | NA | 2.541 | 1.271 |
| Talc | NA | 2.800 | 2.000 |
| Total | | 284.6 | 193.7 |
| Hard gelatin capsule, gray | Size 1 | 1 capsule | 1 capsule |

NA = not applicable.

Formulation C is a membrane controlled system comprising a combination of 30% IR beads, 30% delayed release form using a pH 6.0 enteric coating (DR6.0 beads), and 40% DR6.8 beads as capsule-fill, and characterized by a three-pulsatile dissolution profile. The composition of the DR6.0 beads is shown in Table 11C below.

TABLE 11C
Composition of DR6.0 beads.

| Ingredient | % Content | Range (%) |
|---|---|---|
| Febuxostat | 25.2 | 20-30 |
| Sugar sphere | 41.8 | 40-50 |
| HPMC | 13.0 | 10-16 |
| Methacrylic Acid Copolymer Type A | 18.0 | 13-20 |
| Triethyl citrate | 2.0 | 1-3 |
| Isopropyl alcohol* | — | |
| Water* | — | |

*Removed during processing

The composition of an 80 mg febuxostat Formulation C capsule is shown in Table 11D below.

Table 11D
Composition of 80 mg Formulation
C Capsule (3-Pulse IR + 30DR6.0 + 30DR 6.8).

| | Unit composition |
|---|---|
| Ingredient | (mg/capsule) |
| IR Beads (315 milligrams active (“mgA”)/g) | 76.2 |
| Febuxostat | 24.0 |
| Sugar spheres | 39.8 |
| Hypromellose | 12.4 |
| Talc | 0.8 |
| DR 6.0 Beads (252 mgA/g) | 95.2 |
| Febuxostat | 24.0 |
| Sugar spheres | 39.8 |
| HPMC | 12.4 |
| Methacrylic Acid Copolymer Type A | 17.1 |
| Triethyl citrate | 1.9 |
| Talc | 1.0 |
| DR 6.8 Beads (252 mgA/g) | 127.0 |
| Febuxostat | 32.0 |
| Sugar spheres | 53.1 |
| HPMC | 16.5 |
| Methacrylic Acid Copolymer Type A | 5.7 |
| Methacrylic Acid Copolymer Type B | 17.4 |
| Triethyl citrate | 2.3 |
| Talc | 1.2 |
| Empty capsule | 74.0 |

Formulation D is a membrane controlled system comprising a combination of 30% IR beads and 70% of a delayed-controlled release form as capsule-fill. The delayed-controlled release form comprises a combination of pulsatile and continuous release beads containing a portion of delayed-controlled release beads which contain febuxostat coated with a controlled release layer (“CR-short” beads), which is further coated with a delayed release coating designed to release at pH 6.8 (“DCR6.8” beads). The compositions of the CR-short and DCR6.0 beads are shown in Table 11D below.

Controlled release beads are IR beads coated with a polymer, or mixture of polymers, that decreases the release rate of the drug from the bead, so that the drug is released over an extended period of time. The difference between controlled release beads and delayed release beads is that the release from CR beads is continuous after exposed to dissolution medium over a period of time, whereas release from DR beads is very rapid when the beads are exposed to a pH above which the coating polymer is soluble. Delayed controlled release beads combine the DR and CR concepts with the goal of delaying drug release until the beads are exposed to a pH greater than the pH at which the polymer dissolves and drug release after that point is prolonged over an extended period.

The CR-short beads are designed to complete drug release in 4-6 hours. The composition of the CR-short beads are shown in Table 11E below.

TABLE 11E
Compositions of CR-short beads.

| Ingredient | % Content | Range (%) |
|---|---|---|
| Febuxostat | 22.05 | 20-24 |
| Sugar sphere | 36.58 | 30-40 |
| Hypromellose (in IR bead) | 11.38 | 9-13 |
| Surelease** | 21.0 | 18-24 |
| Hypromellose (in CR coat) | 9.0 | 7-11 |
| Water* | — | |

**Surelease is a ready to use plasticized coating system containing ethylcellulose, dibutylsebacate, oleic acid and silicon dioxide
*Removed during processing

The outer layer of the and DCR6.8 beads dissolves when exposed to a dissolution medium of pH ≧6.8 and the controlled release layer allows dissolution of the drug over a period of 4-6 hours. The composition of the DCR6.8 beads are shown in Table 11F below.

TABLE 11F
Composition for DCR 6.8 beads

| Ingredient | % Content | Range (%) |
|---|---|---|
| Febuxostat | 21.4 | 15-25 |
| Sugar sphere | 35.5 | 30-40 |
| Hypromellose (in IR bead) | 11.1 | 8-14 |
| Surelease E-7-19010 (solid content) | 8.4 | 6.0-10.0 |
| Hypromellose (in CR coat) | 3.6 | 2.0-5.0 |
| Methacrylic Acid Copolymer Type A | 4.6 | 3.0-6.0 |
| Methacrylic Acid Copolymer Type B | 13.6 | 10.0-16.0 |
| Triethyl citrate | 1.8 | 1.0-3.0 |
| Water* | | |
| Acetone* | — | |

*Removed during processing

Drug release from Formulation D is characterized by a 2-pulse release, the first pulse from the IR beads, with a pH-dependent delayed release of a second pulse over 4-6 hours. The composition of an 80 mg febuxostat Formulation D capsule is shown in Table 11G below.

TABLE 11G
Composition of 80 mg Formulation D Capsule (2-Pulse + DCR 6.8).

| | Unit composition |
|---|---|
| Ingredient | (mg/capsule) |
| IR Beads (315 mgA/g) | 72.9 |
| Febuxostat | 24.0 |
| Sugar spheres | 39.8 |
| Hypromellose | 12.4 |
| Talc | 0.8 |
| DCR 6.8 Beads (214.2 mgA/g) | 261.6 |
| Febuxostat | 56.0 |
| Sugar spheres | 92.9 |
| Hypromellose (from IR Beads) | 28.9 |
| Surelease | 22.0 |
| Hypromellose (in coating) | 9.4 |
| Methacrylic Acid Copolymer Type A | 11.9 |
| Methacrylic Acid Copolymer Type B | 35.7 |
| Triethyl citrate | 4.8 |
| Talc | 2.6 |
| Empty capsule | 74.0 |

Formulation E is a membrane controlled system comprising a combination of 20% IR beads and 80% of continuous release beads containing febuxostat coated with a polymeric coating to release the drug over a period of time (“CR-long” beads). The composition of the CR-long beads is shown in Table 11H below.

TABLE 11H
Compositions of CR-long beads.

| Ingredient | % Content | Range (%) |
|---|---|---|
| Febuxostat | 29.6 | 25-35 |
| Sugar sphere | 49.1 | 40-60 |
| Hypromellose (in IR bead) | 15.3 | 12-18 |
| Ethylcellulose | 3.6 | 2.0-5.0 |
| Hypromellose (in CR coat) | 2.4 | 1.0-4.0 |
| Isopropyl alcohol* | — | |
| Water* | — | |

*Removed during processing

The composition of an 80 mg febuxostat Formulation E capsule is shown in Table 11I below. Drug release from Formulation E is characterized by a 2-pulse release.

TABLE 11I
Composition of 80 mg Formulation E Capsule (2-Pulse IR + CR).

| | Unit composition |
|---|---|
| Ingredient | (mg/capsule) |
| IR Beads (315 mgA (milligrams active)/g) | 50.8 |
| Febuxostat | 16.0 |
| Sugar spheres | 26.5 |
| Hypromellose | 8.3 |
| Talc | 0.5 |
| CR Long Beads (296.1 mgA/g) | 216.1 |
| Febuxostat | 56.0 |
| Sugar spheres | 92.9 |
| HPMC | 28.9 |
| Methacrylic Acid Copolymer Type A | 10.0 |
| Methacrylic Acid Copolymer Type B | 30.4 |
| Triethyl citrate | 4.0 |
| Talc | 2.2 |
| Empty capsule | 74.0 |

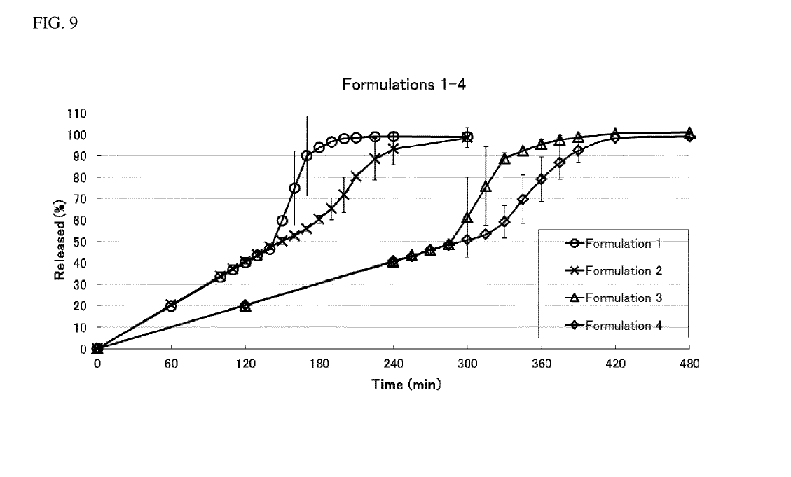

Dissolution data was measured for the febuxostat XR (both 40 and 80 mg febuxostat), Formulations B, C, D, and E using a USP Apparatus I, at 100 rpm, 900 mL of 50 mM phosphate buffer pH 6.90 (the febuxostat XR, Formulations B, C, and D) or pH 7.20 (Formulation E) at 37° C., with manual sampling with medium replacement, and assaying for the drug by HPLC. Dissolution results are shown in FIG. 8.

Some examples of the matrix system formulations are described.

Formulation 1: The matrix system, more specifically as one of the modified release dosage forms, more specifically in the case of matrix system with immediate release core as the matrix system

Formulation 2: The matrix system, more specifically as one of the modified release dosage forms, more specifically in the case of matrix system with sustained release core as the matrix system

Formulation 3: The matrix system, more specifically as one of the modified release dosage forms, more specifically in the case of matrix system (lower release rate than Formulations 1 and 2) with immediate release core as the matrix system

Formulation 4: The matrix system, more specifically as one of the modified release dosage forms, more specifically in the case of matrix system (lower release rate than Formulations 1 and 2) with sustained release core as the matrix system.

Manufacturing Method of Formulation 1
Febuxostat 247.5 g, Lactose monohydrate 577.2 g, Partly pregelatinized starch 150.5 g, HPC-SL 24.7 g, Food Blue No. 1 0.1 g
The above-described raw materials were mixed homogeneously, and the mixture was granulated by fluidized bed granulation, then dried, and subjected to particle size regulation. To 97.0% (w/w) of the powder obtained, 2.0% (w/w) of croscaimellose sodium and 1.0% (w/w) of magnesium stearate were added and mixed. The mixture was subjected to tabletting by a rotary tabletting machine (HT-AP6SS-U; Hata Iron Works Co., Ltd.) under a compression force of about 550 kg to obtain inner cores (diameter: 6 mm, thickness: 3.2 mm), each tablet having a mass of 100 mg.
Febuxosta 140 g, METOLOSE 90SH-100SR 400 g, Lactose monohydrate 435 g, HPC-SL 25 g
The above-described raw materials were mixed, and the mixture was granulated by wet agitation granulation, then dried, and subjected to particle size regulation. To the powder obtained, 0.5% (w/w) of magnesium stearate was added and mixed. This composition was used for the outer layer portion, and tabletted together with the inner core previously prepared by a dry coater tablet press (Libra 45DC; Kikusui Seisakusho Ltd.) under a compression force of about 1 ton to obtain coat-core tablets (diameter: 10 mm, thickness: 6.5 mm), each tablet having a mass of 502 mg and containing 80 mg of Febuxostat.

Manufacturing Method of Formulation 2
Febuxostat 240 g (Average particle diameter: 1.5 .mu.m), Carboxy vinyl polymer 100 g, Lactose monohydrate 634.9 g, HPC-SL 25 g, Food Blue No. 1 0.1 g
The above-described raw materials were mixed homogenously, granulated by wet agitation granulation, then dried, and subjected to particle size regulation. To the powder obtained, 0.5% (w/w) of magnesium stearate was added and mixed. The mixture was tableted by a rotary tabletting machine (HT-AP6SS-U; Hata Iron Works Co., Ltd.) under a compression force of about 350 kg to obtain inner cores (diameter: 6 mm, thickness: 3.4 mm), each tablet having a mass of 100.5 mg.
Febuxostat 140 g, METOLOSE 90SH-100SR 400 g, Lactose monohydrate 435 g HPC-SL 25 g
The above-described raw materials were mixed homogenously, granulated by wet agitation granulation, then dried, and subjected to particle size regulation. To the powder obtained, 0.5% (w/w) of magnesium stearate was added and mixed. This composition was used for the outer layer portion and tableted together with the inner core previously prepared by a dry coater tablet press (Libra 45DC; Kikusui Seisakusho Ltd.) under a compression force of about 1 ton to obtain coat-core tablets (diameter: 10 mm, thickness: 6.5 mm), each tablet containing 80 mg of Febuxostat and having a mass of 502.5 mg.

Manufacturing Method of Formulation 3
Febuxostat 247.5 g, Lactose monohydrate 577.2 g, Partly pregelatinized starch 150.5 g, HPC-SL 24.7 g Food Blue No. 1 0.1 g
The above-described raw materials were mixed homogenously, granulated by fluidized bed granulation, then dried, and subjected to particle size regulation. To 97.0% (w/w) of the powder obtained, 2.0% (w/w) of croscarmellose sodium and 1.0% (w/w) of magnesium stearate were added and mixed. The mixture was tableted by a rotary tabletting machine (HT-AP6SS-U; Hata Iron Works Co., Ltd.) under a compression force of about 550 kg to obtain inner cores (diameter: 6 mm, thickness: 3.2 mm), each tablet having a mass of 100 mg.
Febuxostat 140 g, METOLOSE 90SH-100SR 200 g, METOLOSE 90SH-4000SR 200 g, Lactose monohydrate 435 g, HPC-SL 25 g
The above-described raw materials were mixed homogenously and granulated by wet agitation granulation, then dried, and subjected to particle size regulation. To the powder obtained, 0.5% (w/w) of magnesium stearate was added and mixed.
This composition was used for the outer layer portion and tableted together with the inner core previously prepared by a dry coater tablet press (Libra 45DC; Kikusui Seisakusho Ltd.) under a compression force of about 1 ton to obtain coat-core tablets (diameter: 10 mm, thickness: 6.5 mm), each tablet containing 80 mg of Febuxostat and having a mass of 502 mg.

Manufacturing Method of Formulation 4
Febuxostat 240 g, Carboxy vinyl polymer 100 g, Lactose monohydrate 634.9 g, HPC-SL 25 g, Food Blue No. 1 0.1 g
The above-described raw materials were mixed homogenously, granulated by wet agitation granulation, then dried, and subjected to particle size regulation. To the powder obtained, 0.5% (w/w) of magnesium stearate was added and mixed. The mixture was tableted by a rotary tabletting machine (HT-AP6SS-U; Hata Iron Works Co., Ltd.) under a compression force of about 350 kg to obtain inner cores (diameter: 6 mm, thickness: 3.4 mm), each tablet having a mass of 100.5 mg.
Febuxostat 140 g, METOLOSE 90SH-100SR 200 g, METOLOSE 90SH-4000SR 200 g, Lactose monohydrate 435 g, HPC-SL 25 g

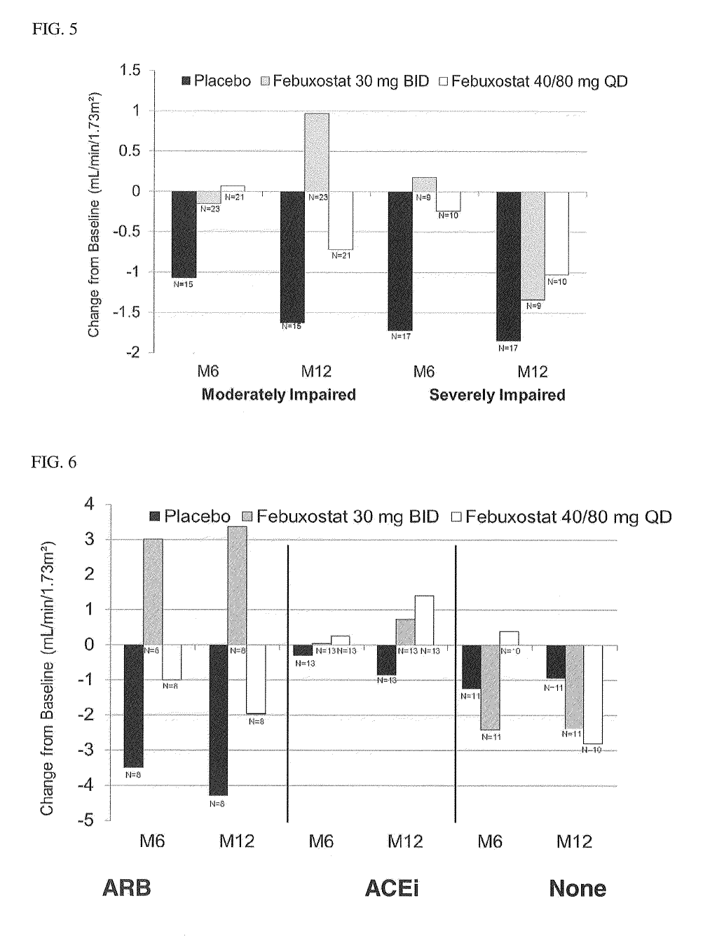

The above-described raw materials were mixed homogenously, granulated by wet agitation granulation, then dried, and subjected to particle size regulation. To the powder obtained, 0.5% (w/w) of magnesium stearate was added and mixed. This composition was used for the outer layer portion and tableted together with the inner core previously prepared by a dry coater tablet press (Libra 45DC; Kikusui Seisakusho Ltd.) under a compression force of about 1 ton to obtain coat-core tablets (diameter: 10 mm, thickness: 6.5 mm), each tablet containing 80 mg of Febuxostat and having a mass of 502.5 mg. Formulation 1, 2, 3, or 4 was subjected to a dissolution test using modified paddle method of the dissolution test of the Japanese Pharmacopoeia with a stationary basket. The conditions of the test were as follows:
Test fluid: 900 mL of diluted Mcllvaine buffer at pH 6.0
Temperature: 37.degree. C.
Number of rotation: 200 rotations/minute Stationary basket: A 40-mesh basket was fixed at the position in the middle between the surface of a test fluid and the bottom of the vessel and about 23 mm from the side wall of a vessel of the dissolution test fluid.
Dissolution results are shown in FIG. 9.

The ratio of Cₘₐₓ/Cₘᵢₙ of febuxostat for a period of from administration to 24 hours was calculated for each formulation. The Cmax/Cmin of formulations B, 1, and 3 at steady state are shown in Table 12 below to have a value of less than or equal to about 50. These values are all below the value of 50 which correlates with the gout flare reduction effect as mentioned in the Example 2.

Dissolution profiles of each representative type of modified release dosage form show that formulations having one of the following in vitro febuxostat dissolution profiles show the gout flare reduction effect:
A: a) 20-60% released after 30 min; b) 70-100% released after 60 min; of the total amount of febuxostat in the dosage form measured in a model system using USP Apparatus 1 operated at pH 6.90 (900 mL of 50 mM phosphate buffer) with stirring at 100 rpm in the case of formulations XR (febuxostat XR), B (Formulation B), C (Formulation C) and D (Formulation D);
B: a) 30-60% released after 60 min; b) 45-75% released after 120 min; c) 70-100% released after 240 min; of the total amount of febuxostat in the dosage form measured in a model system using USP Apparatus 1 operated at pH 7.20 (900 mL of 50 mM phosphate buffer) with stirring at 100 rpm in the case of formulation E;
C: a) 25-55% released after 120-240 min; b) 80-100% released after 180-330 min; of the total amount of febuxostat in the dosage form measured by the dissolution test using modified paddle method of the dissolution test of the Japanese Pharmacopoeia with a stationary basket operated at pH 6.0, 37 degree-C with stirring at 200 rpm in the case of formulations 1 and 3; or
D: a) 25-55% released after 120-240 min; b) 50-70% released after 180-330 min; of the total amount of febuxostat in the dosage form measured by the dissolution test using modified paddle method of the dissolution test of the Japanese Pharmacopoeia with a stationary basket operated at pH 6.0, 37 degree-C with stirring at 200 rpm in the case of the formulations 2 and 4.

### Example 6

### Pharmacokinetic Parameters for Three Modified Release Formulations and an Immediate Release Formulation of Febuxostat at Day 14

This example presents results of pharmacokinetic study of three modified release formulations including 80 mg febuxostat and an immediate release formulation of 80 mg febuxostat at day 14. Various pharmacokinetic parameters determined for the four formulations are summarized in Table 12 below.

In the table below, the four formulations are 1—Formulation 1, 3—Formulation 3, XR—febuxostat XR, and IR—Febuoxostat Immediate Release Formulation.

TABLE 12
Descriptive Statistics for Pharmacokinetic Parameters for Test Products

| For- | | | | | | | | | | | | |
|---|---|---|---|---|---|---|---|---|---|---|---|---|
| mula- | | Tmax | Cmax | Cmax/Dose | C_{24 —}or_Clast | Cmax/ | AUC₂₄ | AUC₀₋₄ | AUC₄₋₂₄ | AUC₂₄/ | MRTlast | MRTINF_obs |
| tion | | (hr) | (ng/ml) | (ng/ml/mg) | (ng/ml) | Cmin | (hr*ng/ml) | (hr*ng/ml) | (hr*ng/ml) | dose | (hr) | (hr) |
| 1 | N | 22 | 22 | 22 | 22 | 22 | 22 | 22 | 22 | 22 | 22 | 22 |
| | Mean | 4.14 | 1399.591 | 17.495 | 34.45 | 50.67 | 5993.877 | 1733.849 | 4260.028 | 74.923 | 7.703 | 9.734 |
| 3 | N | 22 | 22 | 22 | 22 | 22 | 22 | 22 | 22 | 22 | 22 | 22 |
| | Mean | 5.122 | 1011.636 | 12.645 | 59.468 | 21.421 | 5822.715 | 1204.004 | 4618.711 | 72.784 | 9.643 | 14.247 |
| XR | N | 22 | 22 | 22 | 22 | 22 | 22 | 22 | 22 | 22 | 22 | 22 |
| | Mean | 6.069 | 985.182 | 12.315 | 48.705 | 23.448 | 5726.998 | 905.426 | 4821.572 | 71.587 | 9.799 | 12.291 |
| IR | N | 22 | 22 | 22 | 22 | 22 | 22 | 22 | 22 | 22 | 22 | 22 |
| | Mean | 1.823 | 1945.909 | 24.324 | 27.468 | 85.031 | 6895.51 | 3827.418 | 3068.092 | 86.194 | 5.204 | 6.027 |

For comparison, pharmacokinetic parameters from an additional study, the TMX-99-001 study, involving administration of a single dose of various dosage strengths of a febuxostat immediate release formulation are presented below in Table 13 below, along with the results for a twice daily administration of the 30 mg febuxostat IR formulation (designated as “30 mg BID”).

TABLE 13
Pharmacokinetic Parameters for various strengths of a febuxostate immediate release formulation

| | | Tmax | Cmax^{a} | Cmax/dose^{b} | Cmax/ | Tmin | Cmin^{d} |
|---|---|---|---|---|---|---|---|
| Strength | | (hr) | (ng/ml) | (ng/ml/mg) | Cmin^{c} | (hr) | (ng/ml) |
| 30 mg | N | 9 | 9 | 9 | 9 | 9 | 9 |
| | Mean | 0.89 | 1283.54 | 42.78 | 79.40 | 16.89 | 17.50 |
| 40 mg | N | 8 | 8 | 8 | 8 | 8 | 8 |
| | Mean | 1.19 | 1822.11 | 45.55 | 119.45 | 21.00 | 15.13 |
| 50 mg | N | 18 | 18 | 18 | 18 | 18 | 18 |
| | Mean | 1.14 | 1791.71 | 35.83 | 121.77 | 22.00 | 15.98 |
| 70 mg | N | 10 | 10 | 10 | 10 | 10 | 10 |
| | Mean | 1.10 | 2689.91 | 38.43 | 134.12 | 23.20 | 23.81 |
| 90 mg | N | 7 | 7 | 7 | 7 | 7 | 7 |
| | Mean | 1.00 | 4061.63 | 45.13 | 159.22 | 24.00 | 27.53 |
| 120 mg | N | 9 | 9 | 9 | 9 | 9 | 9 |
| | Mean | 1.11 | 5307.58 | 44.23 | 149.48 | 24.00 | 41.49 |
| 160 mg | N | 10 | 10 | 10 | 10 | 10 | 10 |
| | Mean | 0.80 | 8771.07 | 54.82 | 143.92 | 22.80 | 79.88 |
| 180 mg | N | 7 | 7 | 7 | 7 | 7 | 7 |
| | Mean | 1.00 | 8048.76 | 44.72 | 128.34 | 24.00 | 75.73 |
| 240 mg | N | 8 | 8 | 8 | 8 | 8 | 8 |
| | Mean | 0.94 | 11263.03 | 46.93 | 213.76 | 24.00 | 71.30 |
| 30 mg | N | 10 | 10 | 10 | 10 | 10 | 10 |
| BID | Mean | 0.70 | 1488.00 | 49.60 | 28.16 | 24.00 | 59.58 |

^{a}For BID dose, Cmax following AM dose.
^{b}For BID dose, Cmax following AM dose/AM dose (i.e. 30 mg).
^{c}For BID dose, Cmax following AM dose and Cmin from terminal phase at or before 12 hours following PM dose.
^{d}For QD dose, minimum concentration in terminal phase at or before 24 hours. For BID dose, minimum concentration in terminal phase of PM dose.

| | | AUC₀₋₄^{a} | AUC₄₋₂₄^{b} | AUC₂₄^{b} | AUC₂₄/ | MRTINF |
|---|---|---|---|---|---|---|
| | | hr*ng/mL | hr*ng/mL | hr*ng/mL | Dose^{c} | hr |
| 30 mg | N | 9 | 9 | 9 | 9 | 9 |
| | Mean | 1895.79 | 628.87 | 2524.66 | 84.16 | 4.99 |
| 40 mg | N | 8 | 8 | 8 | 8 | 8 |
| | Mean | 3354.02 | 941.11 | 4295.13 | 107.38 | 4.54 |
| 50 mg | N | 18 | 18 | 18 | 18 | 18 |
| | Mean | 3116.06 | 1258.29 | 4374.36 | 87.49 | 5.00 |
| 70 mg | N | 10 | 10 | 10 | 10 | 10 |
| | Mean | 5277.65 | 1670.56 | 6948.21 | 99.26 | 5.00 |
| 90 mg | N | 7 | 7 | 7 | 7 | 7 |
| | Mean | 6893.00 | 2499.81 | 9392.82 | 104.36 | 5.81 |
| 120 mg | N | 9 | 9 | 9 | 9 | 9 |
| | Mean | 8591.01 | 3368.92 | 11959.93 | 99.67 | 5.43 |
| 160 mg | N | 10 | 10 | 10 | 10 | 10 |
| | Mean | 16441.58 | 5840.54 | 22282.12 | 139.26 | 4.65 |
| 180 mg | N | 7 | 7 | 7 | 7 | 7 |
| | Mean | 16809.48 | 7145.06 | 23954.54 | 133.08 | 5.53 |
| 240 mg | N | 8 | 8 | 8 | 8 | 8 |
| | Mean | 25076.83 | 9899.44 | 34976.26 | 145.73 | 4.32 |
| 30 mg | N | 10 | 10 | 10 | 10 | 10 |
| BID | Mean | 2241.00 | 3982.00 | 6224.00 | 103.73 | 18.59 |

^{a}For BID dose, AUC₀₋₄ following AM dose.
^{b}For BID dose, AUC₄₋₂₄ and AUC₂₄ following AM and PM dose.
^{c}For BID dose, AUC₂₄ following AM and PM dose/AM + PM dose (i.e. 60 mg)

As can be seen in the tables above, immediate release formulations, regardless of dose have distinctly different PK profiles than modified release formulations.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”) unless otherwise noted. The modifier “about” used in connection with a quantity is inclusive of the stated value and has the meaning dictated by the context (e.g., includes the degree of error associated with measurement of the particular quantity) or includes values slightly outside the cited values, for example values equaling the cited value plus or minus 10%.

Recitation of ranges of values herein are merely intended to serve as a shorthand method of referring individually to each separate value falling within the range, unless otherwise indicated herein, and each separate value is incorporated into the specification as if it were individually recited herein. The endpoints of all ranges directed to the same component or property are inclusive and independently combinable.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs. The terms wt %, weight percent, percent by weight, etc. are equivalent and interchangeable. The modifier “about” used in connection with a quantity is inclusive of the stated value and has the meaning dictated by the context (e.g., includes the degree of error associated with measurement of the particular quantity).

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a gout patient and reducing the number of gout flares experienced by the patient, the method comprising
administering to a gout patient with hyperuricemia an effective amount of a xanthine oxidase inhibitor in a modified release dosage form once daily or in an immediate release dosage form two or more times daily to reduce the number of gout flares experienced by the patient,
wherein the xanthine oxidase inhibitor is
febuxostat.

2. The method of claim 1, wherein during xanthine oxidase inhibitor administration the number of gout flares characterizing once daily administration of the modified release dosage form or twice daily administration of the immediate release dosage form of the xanthine oxidase inhibitor is reduced from the number of gout flares characterizing once daily administration of an immediate release dosage form of the xanthine oxidase inhibitor.

3. The method of claim 1, wherein the once daily administration of the modified release dosage form or the twice daily administration of the immediate release dosage form produces equivalent or similar serum urate reduction efficacy as once daily administration of an immediate release dosage form.

4. The method of claim 1, wherein during xanthine oxidase inhibitor administration the number of gout flares characterizing once daily administration of the modified release dosage form or twice daily administration of the immediate release dosage form of the xanthine oxidase inhibitor is less than or equal to the number of gout flares characterizing administration of placebo.

5. The method of claim 1, wherein the patient has acute gouty arthritis, chronic gouty joint disease, tophaceous gout, uric acid nephropathy, or nephrolithiasis.

6. (canceled)

7. The method of claim 1, wherein administration of the modified release dosage form once daily or of the immediate release dosage form two or more times daily provides a ratio of a maximum plasma xanthine oxidase inhibitor concentration (Cₘₐₓ) to a minimum plasma xanthine oxidase inhibitor concentration (Cₘᵢₙ) of less than or equal to 60.

8. The method of claim 1, wherein Cₘₐₓ/Cₘᵢₙ is less than or equal to 50.

9-10. (canceled)

11. The method of claim 1, wherein the febuxostat is in the modified release dosage form.

12. The method of claim 1, wherein the amount of febuxostat in the modified release oral dosage form is 40 mg.

13. The method of claim 1, wherein the total amount of febuxostat in the modified release oral dosage form is 80 mg.

14-16. (canceled)

17. The method of claim 1, wherein about 10% to about 30% of the febuxostat in the modified release dosage form is in an immediate release form and about 90% to about 70% of the febuxostat in the modified release dosage form is in a delayed release form.

18. (canceled)

19. The method of claim 1, wherein the modified release dosage form has an in vitro febuxostat dissolution profile of
a) 20-60% released after 30 min;
b) 70-100% released after 60 min;
of the total amount of febuxostat in the dosage form measured using a USP Apparatus I, at 100 rpm, in 900 mL of 50 mM phosphate buffer pH 6.90.

20-22. (canceled)

23. The method of claim 1, wherein the modified release dosage form provides, after administration of a single dose, a mean residence time (MRTinf) of the febuxostat of at least 7 hours.

24. The method of claim 23, wherein the MRTinf is between about 7 hours and about 16 hours.

25. The method of claim 1, wherein the modified release dosage form provides, after administration of a single dose, a Cmax per dose strength of less than about 20 ng/mL/mg.

26. The method of claim 25, wherein the Cmax per dose strength is between about 11 ng/mL/mg to about 13 ng/mL/mg.

27. The method of claim 1, wherein the modified release dosage form provides, after administration of a single dose, a Cmax in the range of about to about 985 ng/ml to about 1400 ng/ml,

28. The method of claim 1, wherein the modified release dosage form provides, after administration of a single dose, a Tmax in the range of about 2 hours to about 8 hours.

29. (canceled)

30. The method of claim 28, wherein Tmax is about 6 hours.

31. The method of claim 1, wherein the modified release dosage form provides, after administration of a single dose, an area under the curve from time 0 to 4 hours (AUC₀₋₄) of about 900 hr-ng/mL to about 1800 hr-ng/mL.

32. The method of claim 1, wherein the modified release dosage form provides, after administration of a single dose, an area under the curve from time 4 hours to time 24 hours (AUC₄₋₂₄) of about 4200 hr-ng/mL to about 4900 hr-ng/mL.

33. The method of claim 1, the method further comprising:
selecting a modified release oral dosage form of the xanthine oxidase inhibitor instead of an immediate release oral dosage form of the xanthine oxidase inhibitor.

34. The method of claim 1, wherein reducing the number of gout flares experienced by the patient occurs during an initial period of administration of the xanthine oxidase inhibitor.

35. The method of claim 34, wherein the initial period of administration of the xanthine oxidase inhibitor is 6 months.

36. (canceled)

37. The method of claim 1, wherein a prophylactic against gout flares is concomitantly administered to the patient.

38. The method of claim 37, wherein the prophylactic is administered concomitantly for the first six months of administration of the xanthine oxidase inhibitor.

39. The method of claim 37, wherein the prophylactic is 0.6 mg colchicine.

40. (canceled)

41. The method of claim 37, wherein reducing the number of gout flares experienced by the patient occurs during the two month period after cessation of concomitant administration of the prophylactic.

42. The method of claim 37, wherein after cessation of concomitant administration of the prophylactic once daily administration of the modified release dosage form or twice daily administration of the immediate release dosage form of the xanthine oxidase inhibitor is characterized by a number of gout flares that is less than or equal to the number of gout flares characterizing administration of placebo.

43-81. (canceled)

82. The method of claim 1, wherein the gout flares are treatment-initiated gout flares.
